(19) United States
(12) Patent Application Publication
(10) Pub. No.: US 2026/0219190 A1
(43) Pub. Date: Jul. 30, 2026

(54) DETECTING DEGRADATION OF A POLYNUCLEOTIDE BY RAMAN SPECTROSCOPY

(71) Applicants: Igor LEDNEV, Albany, NY (US); Alexander SHEKHTMAN, Albany, NY (US); Lamyaa ALMEHMADI, Albany, NY (US); Sergei REVERDATTO, Albany, NY (US)

(72) Inventors: Igor LEDNEV, Albany, NY (US); Alexander SHEKHTMAN, Albany, NY (US); Lamyaa ALMEHMADI, Albany, NY (US); Sergei REVERDATTO, Albany, NY (US)

(73) Assignee: The Research Foundation for the State University of New York, Albany, NY (US)

(21) Appl. No.: 19/147,615

(22) PCT Filed: Jan. 12, 2024
(86) PCT No.: PCT/US2024/011439

§ 371 Date: Jul. 11, 2025

Related U.S. Application Data
(60) Provisional application No. 63/438,968, filed on Jan. 13, 2023.

(51) Int. Cl. G01N 21/65

(52) U.S. Cl. CPC G01N 21/65

(57) ABSTRACT

Provided is a method of detecting degradation of a polynucleotide, including irradiating the polynucleotide with ultraviolet light having a wavelength within a range of from about 200 nm to about 280 nm, and detecting Raman scattering by the polynucleotide. A wavenumber shift of a Raman band or change in peak intensity of a Raman band may constitute detection of polynucleotide degradation. In an example the polynucleotide is in a lipid nanoparticle.

### CROSS-REFERENCE TO RELATED APPLICATIONS

This application is US National Stage Entrant under 35 U.S.C. § 371 of International Patent Application No. PCT/US2024/011439, filed pursuant to the Patent Cooperation Treaty on Jan. 12, 2024, and claims priority of US Provisional Patent Application 63/438,968, filed Jan. 13, 2023, the entire contents of which applications are incorporated herein by reference in their entireties.

### GOVERNMENT RIGHTS STATEMENT

This invention was made with Government support under grant number GM085006 awarded by the National Institutes of Health. The Government has certain rights in the invention.

### BACKGROUND

The success of mRNA-based COVID-19 vaccines has highlighted the potential of RNA-based therapeutics. However, using RNA-based therapeutics is limited by special storage conditions needed to keep RNA stable. To preserve mRNA functions, manufacturers recommend storing vaccines at low temperatures ranging from −80° C. to −20° C. Due to improper temperature control, this requirement has posed challenges resulting in the loss of approximately half of the vaccines distributed worldwide. Due to the intrinsic instability of mRNA caused by its sensitivity to temperature changes, it is vital to develop techniques that can detect mRNA degradation in mRNA vaccines. The current methods used for assessing the quality of vaccines are summarized in a comprehensive review. The World Health Organization mentioned gel electrophoresis, mRNA sequencing techniques, and chromatographic techniques, among others, as typical methods for RNA integrity testing. However, these methods are labor-intensive, require specialized training and laboratory environments, and are resource-intensive and time-consuming. Notably, these methods cannot be performed in situ and require the destruction of samples before analysis.

The present disclosure is directed to overcoming these and other deficiencies in the art.

### SUMMARY

In an aspect, provided is a method of detecting degradation of a polynucleotide, including irradiating the polynucleotide with ultraviolet light having a wavelength within a range of from about 200 nm to about 280 nm, and detecting Raman scattering by the polynucleotide. In the prior example, the polynucleotide optionally includes RNA. In either of the prior examples, the polynucleotide optionally includes mRNA. In any of the prior examples, the polynucleotide optionally includes one or more of N1-methylpseudouridine substituted for uridine, pseudouridine substituted for uridine, and 5-methylcytidine substituted for cytidine. In any of the prior examples, the polynucleotide optionally includes N1-methylpseudouridine substituted for uridine.

In any of the prior examples, the range optionally is from about 230 nm to about 280 nm, or from about 260 nm to about 270 nm, or the wavelength optionally is about 266. In any of the prior examples, the polynucleotide optionally includes a vaccine, an antisense oligonucleotide, or a micro RNA. In any of the prior examples, the polynucleotide optionally is in a lipid nanoparticle.

In any of the prior examples, the Raman scattering optionally includes a wavenumber shift of one or more Raman band of at least 1 cm⁴, or least 2 cm⁻¹, or at least 3 cm⁻¹. In any of the prior examples, the Raman scattering optionally includes a wavenumber shift of a Raman band at 1580 cm⁻¹ or at 1482 cm⁻¹ or both. In any of the prior examples, the Raman scattering optionally includes a decrease in intensity of one or more Raman band. In any of the prior examples, the Raman scattering optionally includes a decrease in intensity of a Raman band at 1580 cm⁻¹ or at 1482 cm⁻¹ or both.

In another aspect, provided is a method of detecting degradation of a test polynucleotide, including irradiating a control polynucleotide and the test polynucleotide with ultraviolet light having a wavelength within a range of from about 200 nm to about 280 nm, detecting Raman scattering by the control polynucleotide and by the test polynucleotide, and detecting a difference between the Raman scattering by the test polynucleotide and by the control polynucleotide.

In the prior example, the test polynucleotide and the control polynucleotide optionally include RNA. In either prior example, the test polynucleotide and the control polynucleotide optionally include mRNA. In any of the prior examples, the test polynucleotide and the control polynucleotide optionally include one or more of N1-methylpseudouridine substituted for uridine, pseudouridine substituted for uridine, and 5-methylcytidine substituted for cytidine. In any of the prior examples, the test polynucleotide and the control polynucleotide optionally include N1-methylpseudouridine substituted for uridine.

In any of the prior examples, the range optionally is from about 230 nm to about 280 nm, or from about 260 nm to about 270 nm, or the wavelength optionally is about 266.

In any of the foregoing examples, the test polynucleotide and the control polynucleotide optionally include a vaccine, an antisense oligonucleotide, or a micro RNA. In any of the prior examples, the test polynucleotide and the control polynucleotide optionally are in lipid nanoparticles.

In any of the prior examples, the difference between the Raman scattering by the test polynucleotide and by the control polynucleotide optionally includes a wavenumber shift of one or more Raman band of at least 1 cm⁻¹, or at least 2 cm⁻¹, or at least 3 cm⁻¹. In any of the prior examples, the difference between the Raman scattering by the test polynucleotide and by the control polynucleotide optionally includes a wavenumber shift of a Raman band at 1580 cm⁻¹ or at 1482 cm⁻¹ or both. In any of the prior examples the difference between the Raman scattering by the test polynucleotide and by the control polynucleotide optionally includes a decrease in intensity of one or more Raman band. In any of the prior examples, the difference between the Raman scattering by the test polynucleotide and by the control polynucleotide optionally includes a decrease in intensity of a Raman band at 1580 cm⁻¹ or at 1482 cm⁻¹ or both.

In another aspect, provided is a method of detecting degradation of an RNA polynucleotide, including irradiating the polynucleotide with ultraviolet light having a wavelength within a range of from about 260 nm to about 270 nm, and detecting Raman scattering by the polynucleotide. In the prior example, the Raman scattering optionally includes a wavenumber shift of one or more Raman band of at least 2 cm⁻¹. In either of the prior examples, the Raman scattering includes a wavenumber shift of a Raman band at 1580 cm⁻¹ or at 1482 cm⁻¹ or both.

In another aspect, provided is a method of detecting degradation of a test polynucleotide, including irradiating a control polynucleotide and the test polynucleotide with ultraviolet light having a wavelength within a range of from about 260 nm to about 270 nm, detecting Raman scattering by the control polynucleotide and by the test polynucleotide, and detecting a difference between the Raman scattering by the test polynucleotide and by the control polynucleotide. In the prior example, the Raman scattering optionally includes a wavenumber shift of one or more Raman band of at least 2 cm⁻¹. In either of the prior examples, the Raman scattering optionally includes a wavenumber shift of a Raman band at 1580 cm⁻¹ or at 1482 cm⁻¹ or both.

In any of the prior examples, the polynucleotide optionally includes DNA.

### BRIEF DESCRIPTION OF THE DRAWINGS

These and other features, aspects, and advantages of the present disclosure will become better understood when the following detailed description is read with reference to the accompanying drawings, wherein:

FIGS. 1A and 1B show lipid nanoparticle (LNP) aging leads to the loss of ψ-mRNA integrity and functionality. NT: non-transfected cells.

FIGS. 2A and 2B show that degradation of polynucleotides is detectable in Deep-UV resonance Raman spectra, in accordance with aspects of the present disclosure. FIG. 2A shows Deep-UV resonance Raman spectra of the mRNA vaccine model before and after the addition of RNase A, and lipofectamine lipids in buffer without mRNA. Inset on the right shows the band shift of 1482 and 1580 cm⁻¹ before and after RNase A addition. FIG. 2B shows a shift of the Raman bands at 1482 and 1580 cm⁻¹ for the mRNA vaccine model degraded with RNase A and aged up to 7 days. Error bars are estimated based on the spectral alignment stability of the spectrograph and the reproducibility of the calibration procedure. Logarithmic trendline (red line) shows the Raman band shift trends. Microsoft Excel, version 2208 was used to plot the spectral band shift and calculate the logarithmic trendline. Above the spectra in 2B is an illustration of decreased structure resulting from degradation of the polynucleotides and as reflected in changes in the Deep-UV resonance Raman spectra.

FIGS. 3A and 3B illustrate Deep-UV Resonance Raman (DUVRR) spectroscopy of the Moderna vaccine lipids and other components. FIG. 3A shows the DUVRR spectrum of the Moderna mRNA vaccine's lipid components. The compounds were dissolved in water in the presence of 10 mM SDS and 10 mM sodium sulfate. FIG. 3B shows the DUVRR spectrum of the Moderna mRNA vaccine's components with sulphate as an internal standard, excluding the mRNA and the lipids. A spectrum of one of the components, sucrose, was also measured under the same conditions for comparison.

FIGS. 4A and 5B show Deep-UV Resonance Raman spectra of the mRNA vaccine model under two degrading conditions. DUVRR spectra representing two degradation experiments conducted with the model mRNA vaccine system are shown. FIG. 4A shows DUVRR spectra collected before and after, in this non-limiting example, RNA with N1-methylpseudouridine (ψ-mRNA) is degraded with RNase A. FIG. 4B shows DUVRR spectra of ψ-mRNA aged for up to 7 days and probed at different time points. The DUVRR spectrum of the buffer mixed with the lipids used is also shown for comparison.

FIGS. 5A-5E show multiple peak fitting of mRNA vaccine model spectra. FIG. 5A shows an RNase A-degraded ψ-RNA vaccine model spectra, and FIGS. 5B-5E show aged ψ-RNA vaccine model sample after different time points. Multiple peak fitting of mRNA vaccine model spectra (1450-1625 cm⁻¹) using Gaussian peak function. The Raman bands 1482 and 1580 cm⁻¹ are highlighted with blue and red (on the left and the right of each spectrograph), respectively.

FIGS. 6A and 6B show Calculated shift of 1482- and 1580 cm⁻¹ Raman bands based on the multiple peak fitting. The shift of the 1482- and (b) 1580-cm⁻¹ Raman bands (FIGS. 6A and 6B, respectively) based on multiple peak fitting procedure is shown for mRNA vaccine model degraded with RNase A and aged for up to 7 days. Error bars are estimated based on the spectral alignment stability of the spectrograph and the reproducibility of the calibration procedure.¹ Logarithmic trendline (red line) is used to show the Raman band shift trends. Microsoft Excel, version 2208 was used for plotting spectral band shift and for calculating the logarithmic trendline.

### DETAILED DESCRIPTION

This disclosure relates to using deep-UV resonance Raman (DUVRR) spectroscopy as an in situ, direct, label-free, and rapid method for probing polynucleotide degradation. One example includes an RNA vaccine system. DUVRR spectroscopy allows the selective probing of nitrogenous bases in polynucleotides by tuning the excitation laser wavelength within the absorption band of the bases, and is applicable for RNA and DNA, including naturally occurring and synthetic, modified nucleotides. A resonance effect occurs, allowing probing of the nitrogenous bases of polynucleotides with higher sensitivity and selectivity. Compared to non-resonance conditions, the resonance enhancement of the Raman signal can reach up to 10⁶, allowing for a significant reduction in spectral interference from, for example, a lipid-based or other matrix in which a polynucleotide may be encapsulated and a focus on the analysis of the nitrogenous bases. As disclosed herein, DUVRR spectroscopy characterizes degradation of polynucleotides, such as DNA and RNA, at various manufacturing stages and thereafter, including product quality validation and establishment of storage condition parameters, where polynucleotide integrity must be verified, including for, but not limited to, polynucleotides including therapeutic compositions such RNA vaccines, microRNA (miRNA), or other polynucleotide-based therapeutics.

As used herein, the terms “includes,” “including,” “includes,” “including,” “contains,” “containing,” “have,” “having,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter.

As used herein, the singular forms “a”, “an” and “the” include plural referents unless the content clearly dictates otherwise. Thus, for example, reference to “a polynucleotide” includes a mixture of two or more polynucleotides, and the like.

As used herein, the term “about” or “approximately” means within 5% of a given value or range.

As used herein, the term “nucleic acid” or “polynucleotide” means single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H+. NH4+, trialkylammonium, tetraalkylammonium, Mg2+, Na+ and the like. A nucleic acid can be a polynucleotide or an oligonucleotide, including polymers of adenosine, cytidine, guanosine, thymidine, uridine, dexoyribonucleotide analogs thereof, and variants of any of the foregoing. A nucleic acid may be composed entirely of deoxyribonucleotides, entirely of ribonucleotides, or chimeric mixtures thereof. The nucleotide monomer units may include any of the nucleotides described herein, including, but not limited to, naturally occurring nucleotides and nucleotides analogs. Nucleic acid may range in size from a few monomeric units, e.g., 5-40, to several thousands of monomeric nucleotide units. Nucleic acids include, but are not limited to, genomic DNA, eDNA, hnRNA, mRNA, rRNA, tRNA, fragmented nucleic acid, nucleic acid obtained from sub-cellular organelles such as mitochondria or chloroplasts, and nucleic acid obtained from microorganisms or DNA or RNA viruses that may be present on or in a biological sample.

In the context of “polynucleotides,” the terms “variant.” “derivative,” and “modified” as used herein refer to a polynucleotide or a fragment thereof, which has been chemically modified, e.g., by the covalent attachment of any type of molecule to the polynucleotide. For example, but not by way of limitation, a polynucleotide or a fragment thereof can be chemically modified, e.g., by acetylation, phosphorylation, methylation, etc. The variants or derivatives are modified in a manner that is different from naturally occurring or starting nucleotide or polynucleotide, either in the type or location of the molecules attached. Variants or derivatives further include deletion of one or more chemical groups which are naturally present on the nucleotide or polynucleotide. A variant or a derivative of a polynucleotide or a fragment of a polynucleotide can be chemically modified by chemical modifications using techniques known to those of skill in the art, including, but not limited to specific chemical cleavage, acetylation, formulation, etc. Further, a variant or a derivative of a polynucleotide or a fragment of a polynucleotide can contain one or more dNTPs or nucleotide analogs. A polynucleotide variant or derivative may possess a similar or identical function as a polynucleotide or a fragment of a polynucleotide described herein. A polynucleotide variant or derivative may possess an additional or different function compared with a polynucleotide or a fragment of a polynucleotide described herein.

A polynucleotide may include an RNA such as an RNA including one or more modified nucleotide, referred to as modified RNA. A polynucleotide, such as a modified RNA, is a synthetic modified RNA that can be used for expression of a gene of interest. Chemical modifications to a ribonucleotide included in modified RNA may stabilize an RNA molecule, blunt an immune response, or enhance transcription. Additionally, unlike delivery of protein agents directly to a cell, which can activate the immune system, the delivery of modified RNA can be achieved without immune impact. For example, substitution of uridine with pseudouridine (5-ribosyluracil) or N1-methylpseudouridine and cytidine with 5-methylcytidine, respectively, drastically reduces the immune response elicited from exogenous RNA without such substitutions. Such modifications may be included in vaccines or other therapeutic compositions including polynucleotides such as RNA or DNA. Stability and translational efficiency from an RNA molecule may also be increased by including a 3′-0-Me-m7G(5′)ppp(5′)G Anti Reverse Cap Analog (ARCA) at the 5′ end of the RNA molecule. Polynucleotides including any of the foregoing or following modifications are expressly included as examples of polynucleotides as disclosed herein.

A polynucleotide, such as a modified RNA, may encompass an RNA molecule with at least uridine substituted with pseudouridine. A polynucleotide, such as a modified RNA, may encompass an RNA molecule with at least cytidine substituted with 5-methylcytidine. A polynucleotide, such as a modified RNA, may encompass an RNA molecule including the modified nucleoside 5-methylcytidine (5mC). A polynucleotide, such as a modified RNA, may encompass an RNA molecule including the modified nucleoside 2-Thiouridine-5′-Triphosphate (2-thio WU). A polynucleotide, such as a modified RNA, may encompass an RNA molecule with at least the modified nucleoside1-Methylpseudouridine-5′-Triphosphate (1-mψU). A polynucleotide, such as a modified RNA, may encompass an RNA molecule with at least the modified nucleoside N1-methyl-pseudouridine (N1mΨ) substituted for uridine. A polynucleotide, such as a modified RNA, may encompass an RNA molecule wherein at least 5′ triphosphates are removed. A polynucleotide, such as a modified RNA, may encompass an RNA molecule wherein at least a 3′-O-Me-m7G(5′)ppp(5′)G Anti Reverse Cap Analog (ARCA) cap or C₃₂H₄₃N₁₅O₂₄P₄ CleanCap Reagent AG is included in a 5′ untranslated regions of the RNA molecule.

A polynucleotide, such as a modified RNA, may be prepared by in vitro transcription. A polynucleotide, such as a modified RNA, may be in vitro transcribed, e.g., from a linear DNA template using one or more reagents selected from a cap analog, guanosine triphosphate, adenosine triphosphate, cytidine triphosphate, uridine triphosphate, and derivatives thereof. A cap analog may be selected from Anti-Reverse Cap Analog (ARCA) 3′-O-Me-m7G(5′)ppp(5′)G, standard cap analog m7G(5′)ppp(5′)G, unmethylated cap analog G(5′)ppp(5′)G, methylated cap analog for A+1 sites m7G(5′)ppp(5′)A, and unmethylated cap analog for A+1 sites G(5′)ppp(5′)A. In certain examples, a cap analog is Anti-Reverse Cap Analog (ARCA) 3′-O-Me-m7G(5′)ppp(5′)G. According to some examples, modified RNA may be in vitro transcribed from a plasmid template using one or more reagents selected from 3′-O-Me-m7G(5′)ppp(5′)G, guanosine triphosphate, adenosine triphosphate, cytidine triphosphate, N1-methylpseudouridine-5-triphosphate, and any one or more of the aforementioned examples of modified RNA, or others, without limitation and in any combination.

Additional suitable modifications to a modified RNA or mRNA molecule are well known in the art (see, e.g., U.S. Pat. No. 8,278,036 to Kariko et al., U.S. Pat. No. 10,086,043 to Chien et al.; U.S. Patent Application Publication No. 2019/0203226 to Zangi et al.; and U.S. Patent Application Publication No. 2018/0353618 to Burkhardt et al., which are hereby incorporated by reference in their entirety). In some embodiments, the nucleoside that is modified in the modified RNA is a uridine (U), a cytidine (C), an adenine (A), or guanine (G). The modified nucleoside can be, for example, m⁵C (5-methylcytidine), m⁶A (N⁶-methyladenosine), s²U (2-thiouridien), ψ (pseudouridine), or Um (2-O-methyluridine). Some exemplary chemical modifications of nucleosides in a modified RNA molecule may further include, for example and without limitation, pyridine-4-one ribonucleoside, 5-aza-uridine, 2-thio-5-aza uridine, 2-thiouridine, 4-thio pseudouridine, 2-thio pseudouridine, 5-hydroxyuridine, 3-methyluridine, 5-carboxymethyl uridine, 1-carboxymethyl pseudouridine, 5-propynyl uridine, 1-propynyl pseudouridine, 5-taurinomethyluridine, 1-taurinomethyl pseudouridine, 5-taurinomethyl-2-thio uridine, 1-taurinomethyl-4-thio uridine, 5-methyl uridine, 1-methyl pseudouridine, 4-thio-1-methyl pseudouridine, 2-thio-1-methyl pseudouridine, 1-methyl-1-deaza pseudouridine, 2-thio-1-methyl-1-deaza pseudouridine, dihydrouridine, dihydropseudouridine, 2-thio dihydrouridine, 2-thio dihydropseudouridine, 2-methoxyuridine, 2-methoxy-4-thio uridine, 4-methoxy pseudouridine, 4-methoxy-2-thio pseudouridine, 5-aza cytidine, pseudoisocytidine, 3-methyl cytidine, N4-acetylcytidine, 5-formylcytidine, N4-methylcytidine, 5-hydroxymethylcytidine, 1-methyl pseudoisocytidine, pyrrolo-cytidine, pyrrolo-pseudoisocytidine, 2-thio cytidine, 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, 2-aminopurine, 2,6-diaminopurine, 7-deaza adenine, 7-deaza-8-aza adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine. N⁶-(cis-hydroxyisopentenyl) adenosine, 2-methylthio-N⁶-(cis-hydroxyisopentenyl) adenosine. N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthio adenine, 2-methoxy adenine, inosine, 1-methyl inosine, wyosine, wybutosine, 7-deaza guanosine, 7-deaza-8-aza guanosine, 6-thio guanosine, 6-thio-7-deaza guanosine, 6-thio-7-deaza-8-aza guanosine, 7-methyl guanosine, 6-thio-7-methyl guanosine, 7-methylinosine, 6-methoxy guanosine, 1-methylguanosine, N²-methylguanosine, N²,N²-dimethylguanosine, 8-oxo guanosine, 7-methyl-8-oxo guanosine, 1-methyl-6-thio guanosine, N²-methyl-6-thio guanosine, or N²,N²-dimethyl-6-thio guanosine.

In an example, modifications made to a modified RNA are independently selected from 5-methylcytosine, pseudouridine, and 1-methylpseudouridine.

In some embodiments, a modified RNA may include a modified uracil selected from the group consisting of pseudouridine (ψ), pyridine-4-one ribonucleoside, 5-aza uridine, 6-aza uridine, 2-thio-5-aza uridine, 2-thio uridine (s2U), 4-thio uridine (s4U), 4-thio pseudouridine, 2-thio pseudouridine, 5-hydroxy uridine (ho⁵U), 5-aminoallyl uridine, 5-halo uridine (e.g., 5-iodom uridine or 5-bromo uridine), 3-methyl uridine (m³U), 5-methoxy uridine (mo⁵U), uridine 5-oxyacetic acid (cmosU), uridine 5-oxyacetic acid methyl ester (mcmo⁵U), 5-carboxymethyl uridine (cm⁵U), 1-carboxymethyl pseudouridine, 5-carboxyhydroxymethyl uridine (chm⁵U), 5-carboxyhydroxymethyl uridine methyl ester (mchm⁵U), 5-methoxycarbonylmethyl uridine (mcm⁵U), 5-methoxycarbonylmethyl-2-thio uridine (mcm⁵s2U), 5-aminomethyl-2-thio uridine (nm⁵s2U), 5-methylaminomethyl uridine (mnm⁵U), 5-methylaminomethyl-2-thio uridine (mnm⁵s2U), 5-methylaminomethyl-2-seleno uridine (mnm⁵se²U), 5-carbamoylmethyl uridine (ncm⁵U), 5-carboxymethylaminomethyl uridine (cmnm⁵U), 5-carboxymethylaminomethyl-2-thio uridine (cmnm⁵s2U), 5-propynyl uridine, 1-propynyl pseudouridine, 5-taurinomethyl uridine (∜cm⁵U), 1-taurinomethyl pseudouridine, 5-taurinomethyl-2-thio uridine (^{TM}^{⁵}s2U), 1-taurinomethyl-4-thio pseudouridine, 5-methyl uridine (m⁵U, e.g., having the nucleobase deoxythymine), 1-methyl pseudouridine (m¹ψ), 5-methyl-2-thio uridine (m⁵s2U), 1-methyl-4-thio pseudouridine (m₁s⁴ψ), 4-thio-1-methyl pseudouridine, 3-methyl pseudouridine (m³ψ), 2-thio-1-methyl pseudouridine, 1-methyl-1-deaza pseudouridine, 2-thio-1-methyl-1-deaza pseudouridine, dihydrouridine (D), dihydropseudouridine, 5,6-dihydrouridine, 5-methyl dihydrouridine (m⁵D), 2-thio dihydrouridine, 2-thio dihydropseudouridine, 2-methoxy uridine, 2-methoxy-4-thio uridine, 4-methoxy pseudouridine, 4-methoxy-2-thio pseudouridine, N¹-methyl pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio uridine (inm⁵s2U), α-thio uridine, 2′-O-methyl uridine (Um), 5,2′-O-dimethyl uridine (m⁵Um), 2′-O-methyl pseudouridine (ψm), 2-thio-2′-O-methyl undine (s2Um), 5-methoxycarbonylmethyl-2′-O-methyl uridine (mcm⁵Um), 5-carbamoylmethyl-2′-O-methyl uridine (ncm⁵Um), 5-carboxymethylaminomethyl-2′-O-methyl uridine (cmnm⁵Um), 3,2′-O-dimethyl uridine (m³Um), 5-(isopentenylaminomethyl)-2′-O-methyl uridine (inmUm), 1-thio uridine, deoxythymidine, 2′-F-ara uridine, 2′-F uridine, 2′-OH-ara uridine, 5-(2-carbomethoxy vinyl) uridine, and 5-3-(1-E-propenylamino) uridine.

In some embodiments, a modified RNA may include a modified cytosine selected from the group consisting of 5-aza cytidine, 6-aza cytidine, pseudoisocytidine, 3-methyl cytidine (m³C), N⁴-acetyl cytidine (act), 5-formyl cytidine (f⁵C), N⁴-methyl cytidine (m⁴C), 5-methyl cytidine (m⁵C), 5-halo cytidine (e.g., 5-iodo cytidine), 5-hydroxymethyl cytidine (hm⁵C), 1-methyl pseudoisocytidine, pyrrolo-cytidine, pyrrolo-pseudoisocytidine, 2-thio cytidine (s2C), 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebulanne, 2-methoxy cytidine, 2-methoxy-5-methyl cytidine, 4-methoxy pseudoisocytidine, 4-methoxy-1-methyl pseudoisocytidine, lysidine (k²C), alpha-thio cytidine, 2′-O-methyl cytidine (Cm), 5,2′-O-dimethyl cytidine (m³Cm), N⁴-acetyl-2′-O-methyl cytidine (ac⁴Cm), N⁴,2′-O-dimethyl cytidine (m⁴Cm), 5-formyl-2′-O-methyl cytidine (f⁵Cm), N⁴,N⁴,2′-O-trimethyl cytidine (m⁴₂Cm), 1-thio cytidine, 2′-F-ara cytidine, 2′-F cytidine, and 2′-OH-ara cytidine.

In some embodiments, a modified RNA may include a modified adenine selected from the group consisting of 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyl adenosine (m¹A), 2-methyl adenine (m²A), N⁶-methyl adenosine (m⁶A), 2-methylthio-N⁶-methyl adenosine (ms²m⁶A). N⁶-isopentenyl adenosine (i⁶A), 2-methylthio-N⁶-isopentenyl adenosine (ms²i⁶A), N⁶-(cis-hydroxyisopentenyl) adenosine (io⁶A), 2-methylthio-N⁶-(cis-hydroxyisopentenyl) adenosine (ms²io⁶A), N⁶-glycinylcarbamoyl adenosine (g⁶A), N⁶-threonylcarbamoyl adenosine (t⁶A), N⁶-methyl-N⁶-threonylcarbamoyl adenosine (m⁶t⁶A), 2-methylthio-N⁶-threonylcarbamoyl adenosine (ms²g⁶A), N⁶,N⁶-dimethyl adenosine (m⁶₂A), N⁶-hydroxynorvalylcarbamoyl adenosine (hn⁶A), 2-methylthio-N⁶-hydroxynorvalylcarbamoyl adenosine (ms² hn⁶A), N⁶-acetyl adenosine (ac⁶A), 7-methyl adenine, 2-methylthio adenine, 2-methoxy adenine, alpha-thio adenosine, 2′-O-methyl adenosine (Am), N⁶,2′-O-dimethyl adenosine (m⁶Am) N⁶,N⁶,2′-O-trimethyl adenosine (m⁶₂Am), 1,2′-O-dimethyl adenosine (m¹Am), 2′-O-ribosyl adenosine (phosphate) (Ar(p)), 2-amino-N⁶-methyl purine, 1-thio adenosine, 8-azido adenosine, 2′-F-ara adenosine, 2′-F adenosine, 2′-OH-ara adenosine, and N⁶-(19-amino-pentaoxanonadecyl) adenosine.

In some embodiments, a modified RNA may include a modified guanine selected from the group consisting of inosine (I), 1-methyl inosine (m¹I), wyosine (imG), methylwyosine (mimG), 4-demethyl wyosine (imG-14), isowyosine (imG2), wybutosine (yW), peroxywybutosine (o₂yW), hydroxywybutosine (OHyW), undermodified hydroxywybutosine (OHyWy), 7-deaza guanosine, queuosine (Q), epoxyqueuosine (oQ), galactosyl queuosine (galQ), mannosyl queuosine (manQ), 7-cyano-7-deaza guanosine (preQ₀), 7-aminomethyl-7-deaza guanosine (preQ₁), archaeosine (G⁺), 7-deaza-8-aza guanosine, 6-thio guanosine, 6-thio-7-deaza guanosine, 6-thio-7-deaza-8-aza guanosine, 7-methyl guanosine (m⁷G), 6-thio-7-methyl guanosine, 7-methyl inosine, 6-methoxy guanosine, 1-methyl guanosine (m¹G), N²-methyl-guanosine (m²G), N²,N²-dimethyl guanosine (m²²G N^{2,7}-dimethyl guanosine (m^{2,7}G), N²,N^{2,7}-dimethyl guanosine (m^{2,2,7}G), 8-oxo guanosine, 7-methyl-8-oxo guanosine, 1-methio guanosine, N²-methyl-6-thio guanosine, N²,N²-dimethyl-6-thio guanosine, alpha-thio guanosine, 2′-O-methyl guanosine (Gm), N²-methyl-2′-O-methyl guanosine (m²Gm), N²,N²-dimethyl-2′-O-methyl guanosine (m²₂Gm), 1-methyl-2′-O-methyl guanosine (m¹Gm), N²,7-dimethyl-2′-O-methyl guanosine (m^{2,7}Gm), 2′-O-methyl inosine (1m), 1,2′-O-dimethyl inosine (m¹Im), 2′-O-ribosyl guanosine (phosphate) (Gr(p)), 1-thio guanosine, O-methyl guanosine, 2′-F-ara guanosine, and 2′-F guanosine.

A polynucleotide, such as a modified RNA, may include, for example, a non-natural or modified nucleotide. The non-natural or modified nucleotide may include, for example, a backbone modification, sugar modification, or base modification. The non-natural or modified nucleotide may include, for example, a base modification. In some embodiments, the base modification is selected from the group consisting of 2-amino-6-chloropurine riboside 5′ triphosphate, 2-aminoadenosine 5′ triphosphate, 2-thiocytidine 5′ triphosphate, 2-thiouridine 5′ triphosphate, 4-thiouridine 5′ triphosphate, 5-aminoallylcytidine 5′ triphosphate, 5-aminoallyluridine 5′ triphosphate, 5-bromocytidine 5′ triphosphate, 5-bromouridine 5′ triphosphate, 5-iodocytidine 5′ triphosphate, 5-iodouridine 5′ triphosphate, 5-methylcytidine 5′ triphosphate, 5-methyluridine 5′ triphosphate, 6-azacytidine 5′ triphosphate, 6-azauridine 5′ triphosphate, 6-chloropurine riboside 5′-triphosphate, 7-deazaadenosine 5′ triphosphate, 7-deazaguanosine 5′ triphosphate, 8-azaadenosine 5′ triphosphate, 8-azidoadenosine 5′ triphosphate, benzimidazole riboside 5′ triphosphate, N-methyladenosine 5′ triphosphate, N¹-methylguanosine 5′ triphosphate, N⁶-methyladenosine 5′ triphosphate, 06-methylguanosine 5′ triphosphate, N¹-methyl-pseudouridine 5′ triphosphate, puromycin 5′-triphosphate, and xanthosine 5′ triphosphate. Thus, according to some embodiments, the modified RNA includes N-methyl-pseudouridine 5′ triphosphate.

As used herein, the term “complementary” when used in reference to a polynucleotide is intended to mean a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction may be nucleotide base specific, e.g., A:T, A:U, and G:C, by Watson-Crick and Hoogsteen-type hydrogen bonding. In certain examples, base-stacking and hydrophobic interactions can also contribute to duplex stability.

As used herein, the term “hybridization” refers to the process in which two single-stranded polynucleotides bind non-covalently to form a stable double-stranded polynucleotide. A resulting double-stranded polynucleotide is a “hybrid” or “duplex.”

As used herein, the term “degradation” or decreased “integrity” of a polynucleotide means a disruption of bonds between adjacent nucleotides or variants thereof in a polynucleotide, or disruption of intra-nucleotide bonds within a polynucleotide, such as to affect the primary, secondary, or tertiary conformation of said polynucleotide leading to disruption of the ability to synthesize a polymer (whether another polynucleotide or a polypeptide) having a sequence as it would such a polymer had if transcribed or translated, as the case may be, from the polynucleotide prior to the degradation or lack of integrity. It can also include a disruption of sequence of the polynucleotide (e.g., breakage of one or more inter-nucleotide bond such as to lead to a partial or complete depolymerization, or inter-nucleotide bond such as to alter the chemical structure of the nitrogenous base of the nucleotide) such as to decrease its ability to specifically hybridize with an otherwise complementary or substantially complementary polynucleotide or other partner polynucleotide with which the polynucleotide would otherwise hybridize. Degradation of a polynucleotide can occur due to exposure thereof to environmental conditions such as temperature and other factors that may promote chemical changes within the polynucleotide or exposure to compositions such as enzymes or compounds that may promote such chemical changes.

Degradation of a polynucleotide results in a loss, decrease, or disruption of ability of a polynucleotide to serve as a template for synthesis of another polymer and/or to hybridize with another polynucleotide or noncovalently bond with another factor such as a protein that would otherwise bind to the undegraded polynucleotide by nature of its length and/or sequence. Degradation may be manifest in, as non-limiting examples, depolymerization (e.g., loss of or decrease in an amount of full-length polynucleotide), altered three-dimensional conformation of the polynucleotide (e.g., due to changes in non-covalent bonds between nucleotides of the polynucleotide such as complementary of nitrogenous bases within the polynucleotide), altered sequence of the polynucleotide, a failure or loss of ability to drive the polymerization of polymers encoded by the polynucleotide under conditions that would otherwise have resulted in polymerization thereof (e.g., decreased expression of protein encoded for by an RNA molecule following transfection therewith compared to protein expression levels as seen following transfection with an the polynucleotide when not degraded), decreased hybridization of the polynucleotide to a complementary polynucleotide or partner polynucleotide sufficiently complementary to permit hybridization (e.g., decreased ability of a micro-RNA to hybridize to a target RNA and prevent translation therefrom), etc.

Polynucleotides, including those for use in therapeutic uses such as for in vivo or ex vivo cellular transfection, may be packaged in or as part of nanoparticle vesicles to promote stability, biocompatibility, cellular targeting, avoidance of immunogenicity, cellular uptake, delivery to particular subcellular compartments, reduction of side effects, improved patient tolerance, etc. A nanoparticle is a composition of matter having a nanoscale-dimension size, such as a diameter from about 1 nm t about 100 nm, though may refer to compositions having a larger diameter as well, such as up to 500 nm. A nanoparticle may provide enhanced cellular uptake and stability of a first and second RNA molecule as described herein. Packaging a first and second RNA molecule in or as part of a nanoparticle may protect them from extracellular degradation processes that may otherwise occur following, for example, systemic or other administration of a first and second RNA molecule, thereby increasing cellular uptake by prolonging the time period between administration of the first and second RNA molecule and when they are taken up by a cell.

A nanoparticle may also improve cellular uptake by providing a mechanism for cellular entry, such as fusion of a nanoparticle's membrane with a cellular membrane for delivery of the nanoparticle's payload to an intracellular compartment. A variety of materials are known to be suitable for nanoparticles for intracellular delivery of their payloads such as lipid or phospholipid micelles or liposomes, metal nanoparticles, such as gold, aluminum, iron nanoparticles, polyacrylamide, polyacrylate, or chitosan nanoparticles, a polymer-based nanoparticle such as a poly lactic-co-glycolic nanoparticle, may be used in accordance with the present disclosure, with a first and second RNA molecule packaged in any type of nanoparticle suitable for an intended purpose, synthesized according to standard methods.

A nanoparticle for complexing with or packaging a polynucleotide as disclosed herein may include one or more lipid or lipid-like molecule. Various lipids and similar compositions are known for use in packaging polynucleotides, such as for use in cellular transfection and delivery of therapeutics to recipients. Examples include, without limitation, N-[1-(2,3-dioleyloxy)-propyl]-N,N,N-trimethylammonium chloride (DOTMA), dioleoylphosphatidylethanolamine (DOPE), 3β-[N—(N′,N′-dimethylaminoethane) carbamoyl] cholesterol (DC-Chol), 1,2-dioleoyl-3-(trimethylammonium) propane (DOTAP), 2,3-dioleyloxy-N-[2-spermine carboxamide]ethyl-N,N-dimethyl-1-propanammonium trifluoroacetate (DOSPA), 1,2-dimyristyloxypropyl-3-dimethylhydroxy ethylammonium bromide (DMRIE), 1,2-dioleoyl-3-dimethylammonium propane (DODAP), 1,2-dioleoyl-3-dimethylammonium propane (DODMA), dioctadecylamidoglycylspermine (DOGS), N⁴-Cholesteryl-Spermine (GL67), 1,2-dilinoleyloxy-3-dimethylaminopropane (DLinDMA), 2-[2,2-bis[(9Z,12Z)-octadeca-9,12-dienyl]-1,3-dioxolan-4-yl]-N,N-dimethylethanamine (DLin-KC2-DMA), (6Z,9Z,28Z,31Z)-Heptatriaconta-6,9,28,31-tetraen-19-yl 4-(dimethylamino)butanoate (D-Lin-MC3-DMA), 1,1′-[[2-[4-[2-[[2-[bis(2-hydroxydodecyl)amino]ethyl](2-hydroxydodecvl)amino]ethyl]-1-piperazinyl]ethyl]imino]bis-2-dodecanol (C12-200), N1,N16-didodecyl-4,7,13-tris[3-(dodecylamino)-3-oxopropyl]-4,7,10,13-tetraazahexadecanediamide (98N12-5), 1,2-dioleyl-sn-glycerol-3-phosphatidylcholine (DOPC), 1,2-distearoyl-sn-glycero-3-phosphocholine (DSPC), 1-stearoyl-2-oleoyl-sn-glycero-3-phosphocholine (SOPC), 9-Heptadecanyl 8-((2-hydroxyethyl)[6-oxo-6-(undecyloxy)hexyl]amino)octanoate (SM-102), monomethoxypolyethyleneglycol-2,3-dimyristylglycerol (DMG), cholesterol (Chol), and other lipids and lipid-like molecules, in various combinations suitable for a given application. In some examples, a lipid nanoparticle including one or more polynucleotide may include modifications such as covalent attachment of polymers such as polyethylene glycol (PEGylation), such as to enhance nanoparticle half-life in circulation following administration in vivo. Assessment of degradation of polynucleotides complexed with any of the foregoing, or other lipid or lipid-like molecules, in or as part of a nanoparticles, may be accomplished in accordance with the methodology as disclosed herein. A nanoparticle including a polynucleotide encapsulated by or complexed with one or more of the foregoing lipids and lipid-like compositions, or similar compounds, on their own or in any combination, is referred to herein as a lipid nanoparticle. Polynucleotides “in” a lipid nanoparticle includes, without limitation, examples where polynucleotides are fully encapsulated by one or more lipid in a nanoparticle, and where nanoparticles and lipids may have distributions as part of a lipid nanoparticle that completely or partially overlap amongst each other. Various configurations of lipids and polynucleotides for lipid nanoparticles are known and included herein as examples of a polynucleotide being in a lipid nanoparticle.

DUVRR spectroscopy as disclosed herein may permit detection of degradation of a polynucleotide. Significantly. and as disclosed here, DUVRR spectroscopy may be used for detection of polynucleotide degradation including when a DUVRR spectroscopy is performed on the polynucleotide without requiring separation of the polynucleotide from other compositions with which it may normally be complexed, encapsulated, comingled, mixed, or otherwise combined. Where such combination of a polynucleotide with another composition exists, DUVRR spectroscopy may be performed as disclosed herein while the polynucleotide is so combined, after combination with said other composition has been achieved. This possibility results from the resonance enhancement of the Raman spectrographic signal emitted by polynucleotides when an excitation wavelength within the deep ultraviolet range is used, enhancing detection of Raman signal attributable to a polynucleotide and permitting its differentiation from and detection over any such signal that may be emitted by another composition with which the polynucleotide may be combined. All of the examples of DUVRR spectroscopy of a polynucleotide as disclosed herein expressly include performance of such a method when the polynucleotide may be complexed, encapsulated, comingled, mixed, or otherwise combined with one or more other composition. A non-limiting example includes a polynucleotide for use as a therapeutic included as part of a lipid nanoparticle. Such polynucleotide may be, for example, an RNA vaccine or a microRNA or antisense oligonucleotide or DNA molecule in a lipid nanoparticle.

As disclosed herein, resonance Raman spectroscopy may be used to detect degradation of a polynucleotide. By using an excitation frequency within a deep ultraviolet range (e.g., in a range from about 200 nm to about 280 nm), the resonance elicited from nitrogenous bases of nucleotides enhances Raman spectrographic signal substantially, by a factor of up to 10⁶, reducing spectral interference from other molecular compositions that may be present with the polynucleotide and permitting signal detection attributable to the polynucleotide. Furthermore, and demonstrated herein, changes in deep-UV resonance Raman (DUVRR) spectroscopy signal are evident coincident with degradation of polynucleotides. Obtaining a DUVRR spectroscopic signal of a polynucleotide and identifying features therein, such as Raman band shift, change in signal intensity, or both, as disclosed herein, indicates structural changes in the polynucleotide amounting to its degradation. DUVRR spectroscopy signals obtained according to the examples disclosed herein therefore indicate that the queried polynucleotide lacks fitness for an intended use, such as encoding another polynucleotide or an amino acid, hybridizing to another polynucleotide, etc.

As skilled persons apprehend, a Raman band indicates a difference between the energy of excitation laser photons and energy of such photons scattered by a composition contacted thereby in Raman spectroscopy. This energy difference corresponds to energy of molecular vibrations in the composition and is expressed as a Raman band having a wavenumber, corresponding to the difference. A change in wavenumber of a Raman band detected for a composition, such as a polynucleotide, or a deviation of a Raman band wavenumber for such a composition from an expected or known Raman band wavenumber, referred to herein as a shift of a Raman bad wavenumber, indicates a change in the composition. As disclosed herein, degradation of a polynucleotide may be detected as a shift in a Raman band wavenumber for a polynucleotide analyzed by DUVRR spectroscopy from a Raman band wavelength for an undegraded polynucleotide, whether the Raman band wavelength for the undegraded polynucleotide was known, such as by comparison to a known standard (examples of which are disclosed herein), or is determined by performing DUVRR spectroscopy on a test polynucleotide to detect possible degradation thereof and on a control non-degraded polynucleotide to provide a comparative Raman band wavelength.

In an example, an excitation wavelength for use in DUVRR spectroscopy as disclosed herein may have a wavelength in a range of from about 200 nm to about 280 nm. For example, an excitation wavelength may be within a range of from about 210 nm or about 220 nm or about 230 nm or about 240 nm or about 250 nm or about 260 nm or about 270 nm to about 280 nm. An excitation wavelength may be within a range of from about 215 nm or about 225 nm or about 235 nm or about 245 nm or about 255 nm or about 265 nm or about 275 nm to about 280 nm. An excitation wavelength may be within a range of from about 210 nm to about 220 nm or to about 230 nm or to about 240 nm or to about 250 nm or to about 260 nm or to about 270 nm, or from about 210 nm to about 215 nm or to about 225 nm or to about 235 nm or to about 245 nm or to about 255 nm or to about 265 nm or to about 275 nm. An excitation wavelength may be within a range of from about 220 nm to about 270 nm, or from about 230 nm to about 270 nm, or from about 240 nm to about 270 nm, or from about 250 nm to about 270, or from about 260 nm to about 270 nm. An excitation wavelength may be within a range of from about 215 nm to about 225 nm, or from about 220 nm to about 230 nm, or from about 225 nm to about 235 nm, or from about 230 nm to about 240 nm, or from about 235 nm to about 245 nm, or from about 240 nm to about 250 nm, or from about 245 nm to about 255 nm, or from about 250 nm to about 260 nm, or from about 255 nm to about 265 nm, or from about 265 nm to about 275 nm. An excitation wavelength may be within a range of between any two of the foregoing wavelengths enumerated as a limit of a range. An excitation wavelength may be about 250 nm, about 251 nm, about 252 nm, about 253 nm, about 254 nm, about 25 nm, about 256 nm, about 257 nm, about 258 nm, about 259 nm, about 260 nm, about 261 nm, about 262 nm, about 263 nm, about 264 nm, about 265 nm, about 266 nm, about 267 nm, about 268 nm, about 269 nm, about 270 nm, about 271 nm, about 272 nm, about 273 nm, about 274 nm, about 275 nm, about 276 nm, about 277 nm, about 278 nm, about 279 nm, or about 280 nm.

As disclosed herein, degradation of a polynucleotide may be evident in a wavenumber shift of a Raman band shown in a DUVRR spectrograph. DUVRR spectroscopy of a polynucleotide may evince one or more Raman band (representing an emission wavelength and referred to in terms of a difference between a wavelength emitted and an excitation wavelength, in accordance with Raman spectroscopy standards), with wavenumbers including, but not limited to, 1580 cm⁻¹, 1482 cm⁻, 1335 cm⁻¹, 1248 cm⁻¹, and any combination of two or more of the foregoing. Degradation of a polynucleotide, such as may be evinced from decrease in presence of polynucleotide such as detected in an electrophoresis gel or a blot, or decreased signal from a transcript or translation product from the polynucleotide or decrease in other effect that results from transfection of a cell with an undegraded polynucleotide or in vitro effect or activity of an undegraded polynucleotide, compared to the polynucleotide in a non-degraded or less-degraded state, is reflected in a shift in a Raman band. A shift in wavenumber of at least about 1 cm⁻¹, or at least about 2 cm⁻¹, or at least about 3 cm⁻¹, or at least about 4 cm⁻¹, or at least about 5 cm⁻¹, or more, positive or negative, may indicate a state of degradation of a polynucleotide. A shaft in wavenumber of at about 1 cm⁻¹, or about 2 cm⁻¹, or about 3 cm⁻¹, or about 4 cm⁻¹, or about 5 cm⁻¹, or more, positive or negative, may indicate a state of degradation of a polynucleotide. A shaft in wavenumber of 1 cm⁻¹, or 2 cm⁻¹, or 3 cm⁻¹, or 4 cm⁻¹, or 5 cm⁻¹, or more, positive or negative, may indicate a state of degradation of a polynucleotide.

As disclosed herein, degradation of a polynucleotide may be evident in a change in peak intensity of a Raman band shown in a DUVRR spectrograph. DUVRR spectroscopy of a polynucleotide may evince one or more Raman band having a peak, or highest, intensity or amplitude of emission. Degradation of a polynucleotide, such as may be evinced from decrease in presence of polynucleotide such as detected in an electrophoresis gel or a blot, or decreased signal from a transcript or translation product from the polynucleotide or in vitro effect thereof etc., compared to the polynucleotide in a non-degraded or less-degraded state, is reflected in decrease in peak intensity of a Raman band. For example, a peak intensity of a Raman band decreasing by about 5%, by about 10%, by about 15%, by about 20%, by about 25%, by about 30%, by about 35%, by about 40%, aby about 50%, by about 55%, by about 65%, or more, may be interpreted as a detection of degradation of the polynucleotide.

Degradation of a polynucleotide may be detected as a magnitude of wavenumber shift of one or more Raman band, a magnitude or percentage of change in intensity of peak intensity of a Raman band or by a combination of the two, such as any combination of any two of the foregoing examples of changes in Raman band wavenumber of peak intensity.

In an example, degradation of a polynucleotide may be detected as a change in magnitude of wavenumber of a Raman band, such as by from about 1 cm⁻¹ to about 5 cm⁻¹, as described above. Degradation of a polynucleotide may be detected as a shift of a Raman band seen at 1580 cm⁻¹ in the absence of degradation by 1 cm⁻¹, by 2 cm⁻¹, by 3 cm⁻¹, by 4 cm⁻¹, by 5 cm⁻¹, or more, as a result of degradation. Raman band wavenumber presented in comparison to the excitation wavelength used for DUVRR spectroscopy as disclosed herein means a Raman band wavenumber may be detected in a manner and magnitude irrespective of the excitation wavelength used to elicit the Raman shift. A shift in Raman band wavenumber likewise is independent of wavelength employed for its elicitation. Thus, in a non-limiting example disclosed herein, a DUVRR spectroscopy excitation wavelength of 266 nm is used, with, for example, a Raman band detected from an undegraded polynucleotide with a wavenumber of 1580 cm⁻¹. A shift in wavenumber may be detected for a degraded polynucleotide, with a magnitude of the shift being by 1 cm⁻¹, by 2 cm⁻¹, by 3 cm⁻¹, by 4 cm⁻¹ by 5 cm⁻¹, or more. For example, DUVRR spectroscopy performed on an undegraded polynucleotide (with, in a non-limiting example, an excitation wavelength of 266 nm) may elicit a Raman band with a wavenumber of 1580 cm⁻¹. DUVRR performed on a polynucleotide and detection of a Raman band not at 1580 cm⁻¹ but at 1581 cm⁻¹, or at 1582 cm⁻¹, or at 1583 cm⁻¹, or at 1584 cm⁻¹, or at 1585 cm⁻¹, or at above 1585 cm⁻¹ may indicate degradation of the polynucleotide.

Performance of DUVRR spectroscopy on a control sample, such as a non-degraded polynucleotide, and detection of Raman bands elicited thereby as a base comparison against which a DUVRR spectrograph from a test sample whose state of possible degradation is not known, to determine whether the test polynucleotide has degraded, may be unnecessary in some examples. A Raman band seen at 1581 cm⁻¹, or at 1582 cm⁻¹, or at 1583 cm⁻¹, or at 1584 cm⁻¹, or at 1585 cm⁻¹, or at above 1585 cm⁻¹ instead of at 1585 cm⁻¹, may indicate that the polynucleotide is degraded, without requiring comparison to a Raman spectrograph elicited by a control, non-degraded polynucleotide. Detection of a Raman band of 1580 cm⁻¹ by the test polynucleotide would constitute detection of the lack of degradation of the test polynucleotide.

In an example, degradation of a polynucleotide may be detected as a change in magnitude of wavenumber of a Raman band, such as by from about 1 cm⁻¹ to about 5 cm⁻¹, as described above. Degradation of a polynucleotide may be detected as a shift of a Raman band seen at 1482 cm⁻¹ in the absence of degradation by 1 cm⁻¹ by 2 cm⁻¹, by 3 cm⁻¹, by 4 cm, by 5 cm⁻¹, or more, as a result of degradation. Raman band wavenumber presented in comparison to the excitation wavelength used for DUVRR spectroscopy as disclosed herein means a Raman band wavenumber may be detected in a manner and magnitude irrespective of the excitation wavelength used to elicit the Raman shift. A shift in Raman band wavenumber likewise is independent of wavelength employed for its elicitation. Thus, in a non-limiting example disclosed herein, a DUVRR spectroscopy excitation wavelength of 266 nm is used, with, for example, a Raman band detected from an undegraded polynucleotide with a wavenumber of 1482 cm⁻¹. A shift in wavenumber may be detected for a degraded polynucleotide, with a magnitude of the shift being by 1 cm⁻¹, by 2 cm⁻¹, by 3 cm⁻¹ by 4 cm⁻¹ by 5 cm⁻¹, or more. For example, DUVRR spectroscopy performed on an undegraded polynucleotide (with, in a non-limiting example, an excitation wavelength of 266 nm) may elicit a Raman band with a wavenumber of 1482 cm⁻¹. DUVRR performed on a polynucleotide and detection of a Raman band not at 1482 cm⁻¹ but at 1483 cm⁻¹, or at 1484 cm⁻¹, or at 1485 cm⁻¹, or at 1486 cm⁻¹, or at 1487 cm⁻¹, or at above 1487 cm⁻¹ may indicate degradation of the polynucleotide.

Performance of DUVRR spectroscopy on a control sample, such as a non-degraded polynucleotide, and detection of Raman bands elicited thereby as a base comparison against which a DUVRR spectrograph from a test sample whose state of possible degradation is not known, to determine whether the test polynucleotide has degraded, may be unnecessary in some examples. A Raman band seen at 1483 cm⁻¹, or at 1484 cm⁻¹, or at 1485 cm⁻¹, or at 1486 cm⁻¹, or at 1487 cm⁻¹, or at above 1487 cm⁻¹ instead of at 1482 cm⁻¹, may indicate that the polynucleotide is degraded, without requiring comparison to a Raman spectrograph elicited by a control, non-degraded polynucleotide. Detection of a Raman band of 1482 cm⁻¹ by the test polynucleotide would constitute detection of the lack of degradation of the test polynucleotide.

In an example, degradation of a test polynucleotide may be detected as a change in magnitude of wavenumber of a Raman band, such as by from about 1 cm⁻¹ to about 5 cm⁻¹, as described above, in comparison to a Raman band detected by performing DUVRR on a control polynucleotide sample that has not degraded. Degradation of a test polynucleotide may be detected as a shift of a Raman band seen at 1580 cm⁻¹ in the absence of degradation of the control polynucleotide by 1 cm⁻¹, by 2 cm⁻¹, by 3 cm⁻¹, by 4 cm⁻¹, by 5 cm⁻¹, or more, as a result of degradation as seen in the test polynucleotide. Raman band wavenumber presented in comparison to the excitation wavelength used for DUVRR spectroscopy as disclosed herein means a Raman band wavenumber may be detected in a manner and magnitude irrespective of the excitation wavelength used to elicit the Raman shift. A shift in Raman band wavenumber likewise is independent of wavelength employed for its elicitation. Thus, in a non-limiting example disclosed herein, a DUVRR spectroscopy excitation wavelength of 266 nm is used, with, for example, a Raman band detected from an undegraded control polynucleotide with a wavenumber of 1580 cm⁻¹. A shift in wavenumber may be detected for a test polynucleotide, with a magnitude of the shift being by 1 cm⁻¹, by 2 cm⁻¹, by 3 cm⁻¹, by 4 cm⁻¹, by 5 cm⁻¹, or more, constituting detection of degradation of the test polynucleotide. For example, DUVRR spectroscopy performed on an undegraded control polynucleotide (with, in a non-limiting example, an excitation wavelength of 266 nm) may elicit a Raman band with a wavenumber of 1580 cm⁻¹. DUVRR performed on a test polynucleotide and detection of a Raman band not at 1580 cm⁻¹ but at 1581 cm⁻¹, or at 1582 cm⁻¹, or at 1583 cm⁻¹, or at 1584 cm⁻¹, or at 1585 cm⁻¹, or at above 1585 cm⁻¹ may indicate degradation of the test polynucleotide. Detection of a Raman band of 1580 cm⁻¹ by the test polynucleotide would constitute detection of the lack of degradation of the test polynucleotide.

In an example, degradation of a test polynucleotide may be detected as a change in magnitude of wavenumber of a Raman band, such as by from about 1 cm⁻¹ to about 5 cm⁻¹, as described above, in comparison to a Raman band detected by performing DUVRR on a control polynucleotide sample that has not degraded. Degradation of a test polynucleotide may be detected as a shift of a Raman band seen at 1482 cm⁻¹ in the absence of degradation of the control polynucleotide by 1 cm⁻¹, by 2 cm⁻¹, by 3 cm⁻¹, by 4 cm⁻¹, by 5 cm, or more, as a result of degradation as seen in the test polynucleotide. Raman band wavenumber presented in comparison to the excitation wavelength used for DUVRR spectroscopy as disclosed herein means a Raman band wavenumber may be detected in a manner and magnitude irrespective of the excitation wavelength used to elicit the Raman shift. A shift in Raman band wavenumber likewise is independent of wavelength employed for its elicitation. Thus, in a non-limiting example disclosed herein, a DUVRR spectroscopy excitation wavelength of 266 nm is used, with, for example, a Raman band detected from an undegraded control polynucleotide with a wavenumber of 1482 cm⁻¹. A shift in wavenumber may be detected for a test polynucleotide, with a magnitude of the shift being by 1 cm⁻¹, by 2 cm⁻¹, by 3 cm⁻¹, by 4 cm⁻¹, by 5 cm⁻¹, or more, constituting detection of degradation of the test polynucleotide. For example, DUVRR spectroscopy performed on an undegraded control polynucleotide (with, in a non-limiting example, an excitation wavelength of 266 nm) may elicit a Raman band with a wavenumber of 1482 cm⁻¹. DUVRR performed on a test polynucleotide and detection of a Raman band not at 1482 cm⁻¹ but at 1483 cm⁻¹, or at 1484 cm⁻¹, or at 1485 cm⁻¹, or at 1486 cm⁻¹, or at 1487 cm⁻¹, or at above 1487 cm⁻¹ may indicate degradation of the test polynucleotide. Detection of a Raman band of 1482 cm⁻¹ by the test polynucleotide would constitute detection of the lack of degradation of the test polynucleotide.

Any of the foregoing examples may be applied to a polynucleotide for detection of degradation, or detection of lack of degradation. Upon applying DUVRR spectroscopy on a polynucleotide sample and detecting degradation as disclosed above, the sample of polynucleotide may be determined no longer to be useful for the purpose for which it, as a non-degraded sample, had been intended. Such a sample may be discarded or otherwise separated from undegraded samples or destroyed, or otherwise marked or indicated so as to prevent its use under circumstances when use of a degraded polynucleotide would be undesirable or disadvantageous. As an example, the method may be adopted as a quality control method for ascertaining fitness of a therapeutic polynucleotide, and samples for which degradation is detected may be excluded from the supply of therapeutics, such as, without limitation, RNA vaccines, microRNA-based treatments, etc. Such a quality control process may also be adopted to determine whether or not processing, packaging, storage, manufacturing, nanoparticle encapsulation, or other process or processes to which a polynucleotide is subjected or is contemplated for possible exposure to is desirable. If, after exposure to such a condition, polynucleotide degradation is detected according to the present disclosure and above examples, exposure to such condition may be avoided in future. Or, exposure to such a condition that results in the lest detection of degradation may be adopted for future use to the exclusion of other conditions resulting in detection of higher levels of degradation.

### EXAMPLES

The following examples are intended to illustrate particular embodiments of the present disclosure, but are by no means intended to limit the scope thereof.

### Materials and Methods

### Cells and Reagents

Human embryonic kidney cells (HEK293T) were purchased from ATCC (American Type Culture Collection) and cultured according to the recommended protocol. N1-methylpseudouridine-triphosphate was purchased from TriLink Biotechnologies (San Diego, CA). Transfection reagent Lipofectamine 2000 (L2000), Dulbecco's modified Eagle's medium (DMEM), Opti-MEM reduced serum medium, PBS and RiboRuler High Range RNA Ladder were purchased from ThermoFisher Scientific (Waltham, MA). The plasmid (pTK305) used for in vitro transcription of firefly luciferase encoding mRNA was purchased from Addgene (plasmid #66812; RRID:Addgene_66812).¹⁰ Standard Moderna Lipids reported in Reference (3), SM-102, DSPC, Cholesterol, and DMG-PEG (2000), were purchased from Sigma-Aldrich. Sodium Dodecyl Sulfate (SDS) was purchased from Bio-RAD. Other components of the Moderna vaccine reported in Reference (11) are tris(hydroxymethyl)aminomethane (Tris)-HCl buffer (J. T. Baker), acetic acid (Fisher Scientific), sodium acetate (AMRESCO) and Sucrose (J. T. Baker).

### In Vitro RNA Synthesis

A protocol for in vitro mRNA synthesis was described.¹² Briefly, plasmid pTK305 was amplified, linearized with AscI restriction enzyme (NEB), extracted with phenol/chloroform and precipitated with ethanol/ammonium acetate. Linearized DNA was redissolved in RNase-free TE buffer at concentration of 1 mg/ml and served as a template for in vitro transcription using the MEGAscript® T7 Transcription Kit (Ambion, Austin, TX) with the N1-methylpseudouridine-triphosphate completely replacing uridine-triphosphate in the reaction. RNA synthesis was performed according to manufacturer protocol, and resulting mRNA was purified by precipitation with LiCl/EDTA solution (7.5 M lithium chloride, 50 mM EDTA) for ≥30 min at −20° C. RNA pellet was dissolved in RNase-free water or SH buffer at concentration of 1 mg/ml, aliquoted and stored at −70° C.

### Lipid Nanoparticles Aging

For determination of Luciferase RNA stability by agarose gel and Raman spectroscopy, for each time point 30 μg of Luciferase RNA in SH buffer (120 mM NaCl, 20 mM HEPES pH-7.4) was mixed with 30 μL of the same buffer, 30 μL of 1 M sodium sulfate and 30 μL of Lipofectamine-2000. The total volume of the first and the second sample of the vaccine model was 120 μL and 150 μL, respectively. All solutions were RNAse-free. To produce RNase-free Na₂SO₄, salt was heated at 500° C. for 1 hour using a tube furnace (Lindberg). After incubation at room temperature for predetermined time, 5 μl aliquot for agarose gel was withdrawn from the sample, mixed with 5 units of ScriptGuard™ RNase Inhibitor (Cellscript, LLC) and stored at −70° C. Rest of the sample was immediately subjected to Raman analysis.

For agarose gel analysis of the samples, the technique of “bleach gel” was used.¹³ Briefly, agarose was mixed with 1×TBE buffer to obtain 1% gel, and common household bleach (6% sodium hypochlorite) was added to 1%. Agarose was melted, cooled down and ethidium bromide to approx. 1 μg/mL was added before casting. mRNA samples were mixed with 2×RNA Loading Dye (from RiboRuler kit) and loaded at a rate of 1 μg per well along with 3 μL of ready-to-use RiboRuler High Range RNA Ladder in the control lane. Before loading both samples and RNA ladder were heated at 70° C. for 10 min and then chilled on ice for 3 minutes.

For Luciferase function preservation tests, 1 μg of Luciferase RNA in SH buffer was mixed with 1 μL of the same buffer, 1 μL of 1 M sodium sulfate and 1 μL of Lipofectamine-2000 in 200 μL PCR tube. Samples were incubated for the indicated time intervals at room temperature.

### Cell Transfections and Luciferase Activity Assays

HEK 293T cells were grown in in Dulbecco's modified Eagle's medium (DMEM) supplemented with 4 mM L-glutamine, 5.5 mM D-glucose, 1 mM sodium pyruvate, 10% fetal bovine serum (HyClone, Logan, UT), 100 units/mL penicillin and 100 mg streptomycin/mL For experiments targeted on evaluation of the loss of RNA ability to produce functional Luciferase enzyme upon LNP aging, cells were grown on 10 cm plates to 60-80% confluency and then harvested with 0.25% trypsin-EDTA (Gibco, Amarillo, TX) for 5-10 min at 37° C. Trypsin was neutralized by a 5-fold dilution with complete medium, cells were counted and pelleted by centrifugation at 200 g for 10 minutes. Cells were resuspended in Opti-MEM/5% FBS at the rate of 10⁵ cells per mL, and aliquoted into 1.5 mL Eppendorf tubes at 0.5 mL per tube. Tubes were incubated in the growing chamber in the stand at an angled position with lids opened. For transfection aged LNP mixes were combined with 50 μL of OptiMEM and added to the appropriate 1.5 mL Eppendorf tubes with cells. Tubes were left in the growth chamber (37° C., 5% CO₂) at an angled position with lids opened for 16 hours. Next cells were spun down for 5 min at 300 g, washed with PBS and lysed by addition of 50 μL of 1× Luciferase Cell Culture Lysis Reagent (Promega). Tubes were centrifuged for 5 min at 16000 g, and supernatant (~40 μL) was transferred into 200 μL PCR tubes and stored at −70° C. until assay time. For assays, 5 μL of each lysate was transferred into the wells of 96 well white wall plate. 50 μL of Luciferase Assay Reagent was added, and the plate was immediately read on Synergy H1 Hybrid Multi-Mode plate reader (BioTek Instruments, Winooski, VT). Luminescence data was analyzed by using GraphPad prism v 9.0.2 (GraphPad Software, San Diego, CA).

mRNA Vaccine Model

The mRNA vaccine model is composed of 250 μg/ml of Luciferase RNA and 250 L/ml of Lipofectamine 2000 in the SH buffer. A final concentration of 250 mM of sodium sulfate (Acros Organics) was added as a spectroscopic internal standard.

Two batches of mRNA vaccine model were prepared. In the first experiment, a vaccine batch (total volume 120 μl) was treated with 0.8 ng/μL of RNase A for 20 minutes to degrade the mRNA. The second vaccine batch was aged at room temperature for up to 7 days. The samples were analyzed before and after RNase A addition and at different time points for the aged sample using gel electrophoreses and deep-UV Resonance Raman Spectroscopy to probe and monitor the mRNA degradation in the mRNA vaccine model.

For the Moderna lipids-only sample, the lipids were mixed with the molar ratio indicated (3). Briefly, (heptadecan-9-yl 8-((2-hydroxyethyl) (6-oxo-6-(undecyloxy) hexyl) amino) octanoate, SM-102 (Cayman Chemical), 1,2-distearoyl-sn-glycero-3 phosphocholine, DSPC (Cayman Chemical), Cholesterol (Cayman Chemical), and monomethowpolyethyleneglycol-2,3-dimyristylglycerol with polyethylene glycol of average molecular weight 2000, DMG-PEG (2000) (Cayman Chemical), were mixed with a molar ratio of 50:10:38.5:1.5, respectively. The lipids were initially diluted in water in the presence of 10 mM of Sodium Dodecyl Sulfate (SDS), then ultrasonicated for 30 minutes. The total molar concentration of lipids tested using deep-UV Resonance Raman spectroscopy was 1.0 mM. An internal standard, sodium sulfate, at a concentration of 10 mM was added to the solution for further spectral analysis. The buffer components reported in the Moderna vaccine were also investigated at the same ratio reported in Ref (11) using deep-UV resonance Raman spectroscopy-specifically, Tris-HCl [pH 7.5], acetic acid, sodium acetate, and sucrose, were mixed at concentrations of 20 mM, 1.4 mM, 3 mM, and 254 mM, respectively. Sodium sulfate was added as an internal standard at a concentration of 10 mM. A separate solution containing 254 mM sucrose dissolved in water was prepared and tested for comparison.

Samples were loaded in a Suprasil (NMR) tube (SP-Wilmad-LabGlass) containing a magnetic stir bar for deep-UV Resonance Raman Spectroscopy. The Suprasil tube and the magnetic stir bar were treated against RNase using 0.1% Diethyl Pyrocarbonate (Thermo Scientific).

### Deep-UV Resonance Raman

A Deep-UV Raman spectrograph, described elsewhere (Aranda, P. S.; LaJoie, D. M.; Jorcyk, C. L. Bleach gel: a simple agarose gel for analyzing RNA quality. Electrophoresis 2012, 33 (2), 366-369) was used. Briefly, the 4^{th} harmonic of Nd:YAG laser (266-nm) was used for the excitation. The DUVRR spectra were collected by focusing the UV laser within a Suprasil NMR tube (SP-Wilmad-LabGlass) containing about 150 μL of solution. A magnetic stir bar was used to continuously mix the sample and prevent photodegradation. The scattered light was collected in a backscattering geometry and coupled to a double monochromator optimized for UV and deep UV spectral regions with a CCD camera (Roper Scientific) cooled with liquid nitrogen. A 100 nm slit was selected for spectral resolution and signal intensity.¹⁴ WinSpec32 (Roper Scientific) was used for DUVRR Spectra data collection. The laser power measured at the sample was ~0.5 mW.

Each reported DUVRR spectrum is an average of 10 accumulations, 30 seconds each. The spectra were loaded to GRAMS v9.2 software (ThermoFisher Scientific) and calibrated with Raman spectrum of Teflon standard. The accuracy (+/−0.5 cm⁻¹) of the Raman band position has been estimated based on the spectral alignment stability of the spectrograph and the reproducibility of the calibration procedure. Lednev at al., Deep-UV Raman spectrometer tunable between 193 and 205 nm for structural characterization of proteins. Analytical and Bioanalytical Chemistry 2005, 381 (2), 431-437.

### Data Preprocessing and Visualization

PLS_Toolbox (Eigenvector Research Inc, Wenatchee. WA, USA) operating within MATLAB softvare, version 2017b, Mathworks, Inc, Natick, MA, USA) was used for data analysis and prepossessing. Spectra were preprocessed using baseline correction with automatic weighted least squares (6th-order polynomial), then smoothing. The spectral normalization is based on the area under the SO₄²⁻ band between 965-993 cm⁻¹.

For the multiple peak fitting procedure, the DUVRR spectrum of Lipofectamine lipids in buffer was subtracted from the Raman spectra of the mRNA vaccine model to remove a water contribution. Spectral subtraction was done using GRAMS v9.2 software. Then, the spectra were truncated to the region between 1450-1625 cm⁻¹. OriginPro 2023 software was then used for the multiple peak fitting using Gaussian peak function. Logarithmic trendline of the Raman band shifts of the 1482- and 1580 cm⁻¹ bands was calculated using Microsoft Excel.

### Example I

To investigate the feasibility of DUVRR spectroscopy for probing the mRNA degradation in mRNA vaccines, a realistic mRNA vaccine model system consisting of N1-methylpseudouridine-modified luciferase mRNA, RNA,¹⁵ in sodium-HEPES (SH) buffer encapsulated with lipofectamine lipids was utilized. Sodium sulfate was added to the solution as an internal spectroscopic standard. Two separate experiments were performed. In the first experiment, luciferase RNA was degraded with RNase A and it served as a reference for a degraded vaccine sample. RNase A added at a concentration of 8 ng/μl was not detectable with DUVRR spectroscopy Conversely, in the second experiment, RNA was aged up to 7 days at room temperature while DUVRR spectroscopy and gel electrophoresis were used to monitor its stability. All samples were continuously rotated to prevent photodecomposition while irradiated with 266-nm laser light at ~0.5 mW. Geng, et al. 2017 reported no DNA degradation when samples were irradiated with a 244 nm laser at 3 mW.⁹

In addition, a cell transfection test was performed to evaluate the functionality of the RNA in the aged vaccine model.

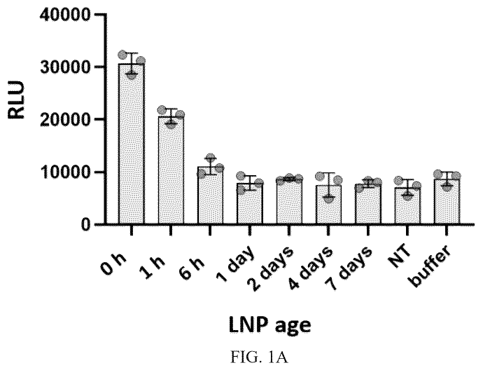

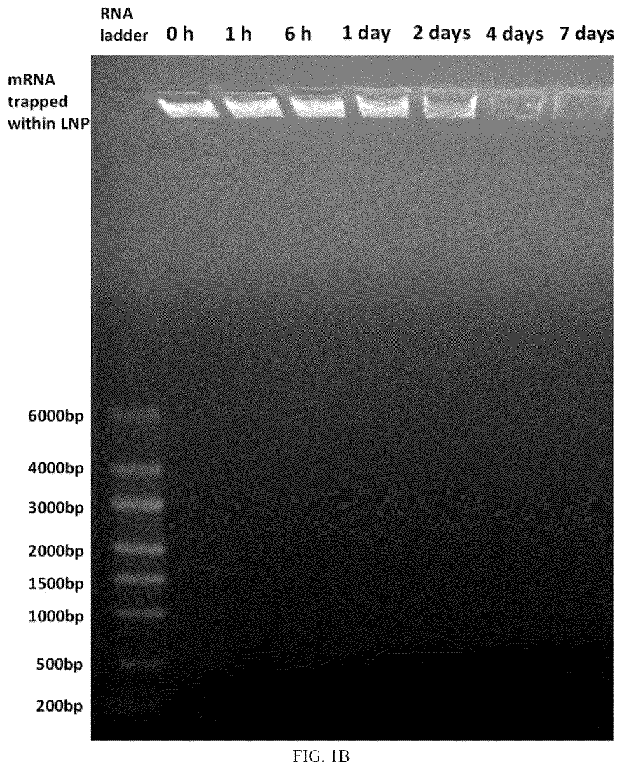

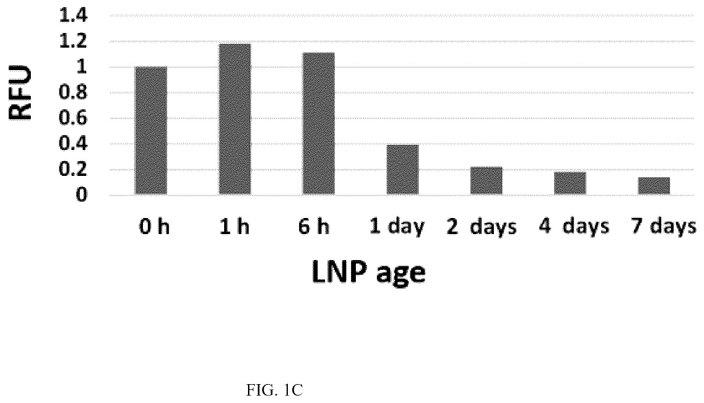

Initially, two tests were conducted to probe the aging-related loss of functionality and integrity of RNA in the vaccine model system. The ability of the RNA transfected into cells to produce functional enzymes decreases continuously as the vaccination model system ages, as depicted in FIG. 1A. Gel electrophoresis further confirmed the RNA degradation as illustrated in FIGS. 1B and 1C.

FIG. 1A illustrates loss of the ability to produce a functional enzyme by luciferase RNA due to LNP aging. RNA was incubated with lipofectamine for different periods and then transfected into HEK293T cells. The cells were harvested after 16 h, and the luminescence produced by cell extracts was recorded. Samples were measured repeatedly three times: the column heights represent the mean, and error bars shown on the graph represent the standard deviation.

FIG. 1B shows an RNA gel, loaded with equal amounts of LNPs of different age and stained with ethidium bromide after the run, show progressive loss of staining due to RNA degradation. Notably, LNPs with RNA do not migrate into the gel¹⁶. FIG. 1C shows quantification of the stained RNA in the gel wells in panel B was carried out using BioRad Image Lab.

### Example 2

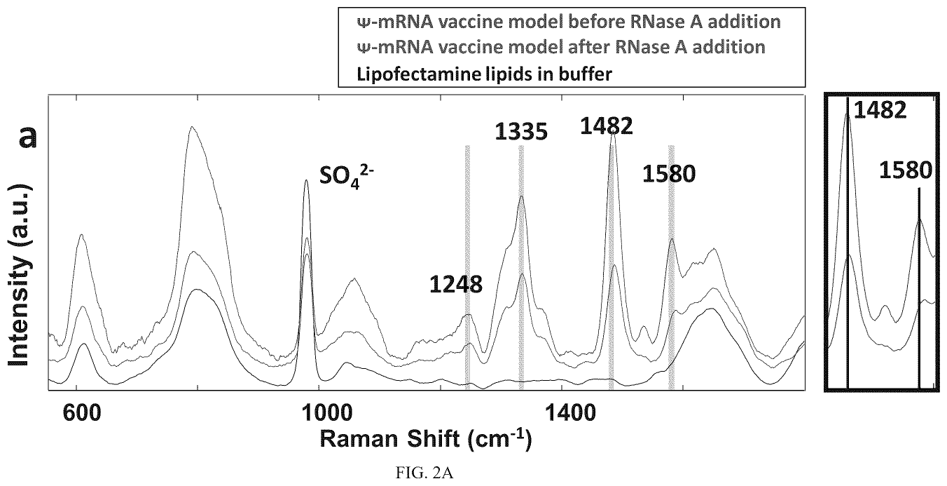

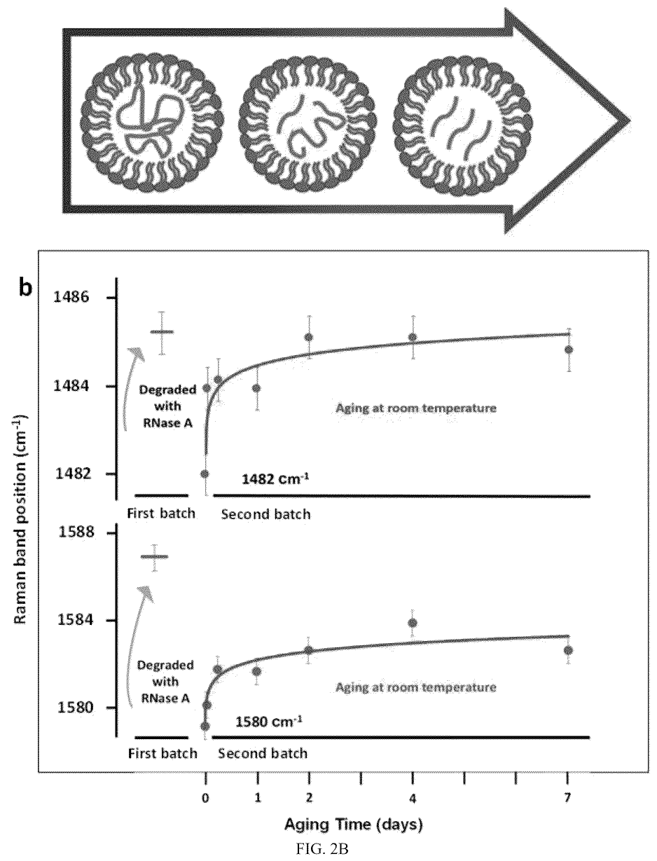

To directly probe RNA in the vaccination model system, DUVRR spectroscopy was used. The choice of 266-nm excitation was based on the resonance enhancement of Raman scattering by RNA (Fodor et al., Ultraviolet resonance Raman spectroscopy of the nucleotides with 266-, 240-, 218-, and 200-nm pulsed laser excitation. Journal of the American Chemical Society 1985, 107 (6), 1520-1529.) and the minimal contribution of other components of a typical vaccine formulation. FIG. 2A shows the DUVRR spectra of the mRNA vaccination model and the lipofectamine lipids in the buffer without RNA. The signal from RNA dominates the DUVRR spectrum of the mRNA vaccine model, with no noticeable contribution from lipids. The resonance Raman spectra of RNA in the vaccine model system are consistent with the previously reported spectra of nitrogenous bases obtained under 266 nm excitation. Fodor et al., Ultraviolet resonance Raman spectroscopy of the nucleotides with 266-, 240-, 218-, and 200-nm pulsed laser excitation. Journal of the American Chemical Society 1985, 107 (6), 1520-1529; Fodor et al., Ultraviolet resonance Raman spectroscopy of DNA with 200-266-nm laser excitation. Journal of the American Chemical Society 1986, 108 (12), 3198-3205; Rossi et al., Insight into the thermal stability of DNA in hydrated ionic liquids from multi-wavelength UV resonance Raman experiments. Physical Chemistry Chemical Physics 2021, 23 (30), 15980-15988. Raman bands at 1580 and 1482 cm⁻¹ correspond to adenine and guanine, respectively. Furthermore, the Raman bands at 1335 and 1248 cm⁻¹ are assigned to adenine and cytosine, respectively. Table I lists the vibrational modes corresponding to each Raman band.

TABLE 1
RNA Raman bands

| Raman band (cm⁻¹) | Assignment |
|---|---|
| 1580 | Adenine: C5═C4 and C4—N3 |
| | Guanine: C4—N3, C5═C4, N7—C5 |
| 1482 | Adenine: C2—H, N9—C8, and C8—H |
| | Guanine: C8—H, N9—C8, and C8═N7 |
| 1335 | Adenine: N7—C5 and C8═N7 |
| 1248 | Cytosine: C6—H and C4—N4 |

### Structures of Adenine, Guanine, and Cytosine (Numbered) Attached to the Ribose Group:

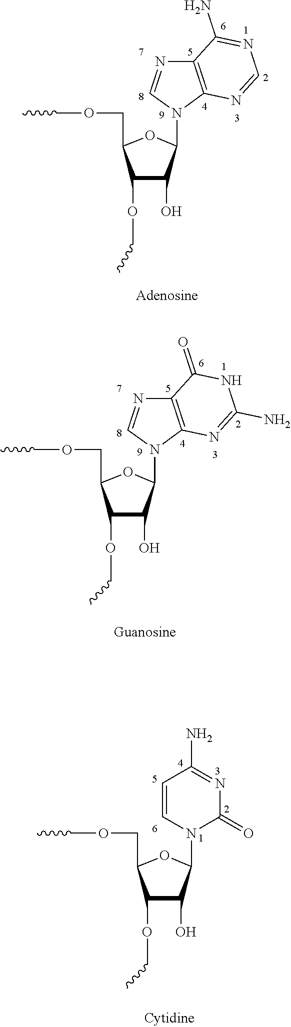

### Structure of Uracil (Numbered):

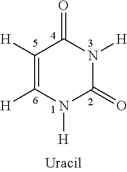

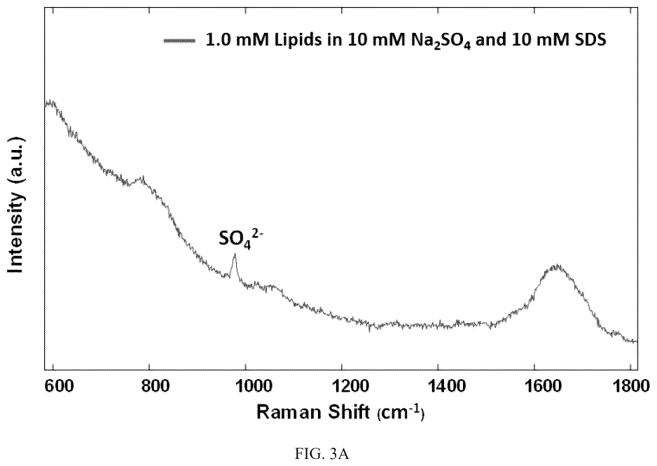

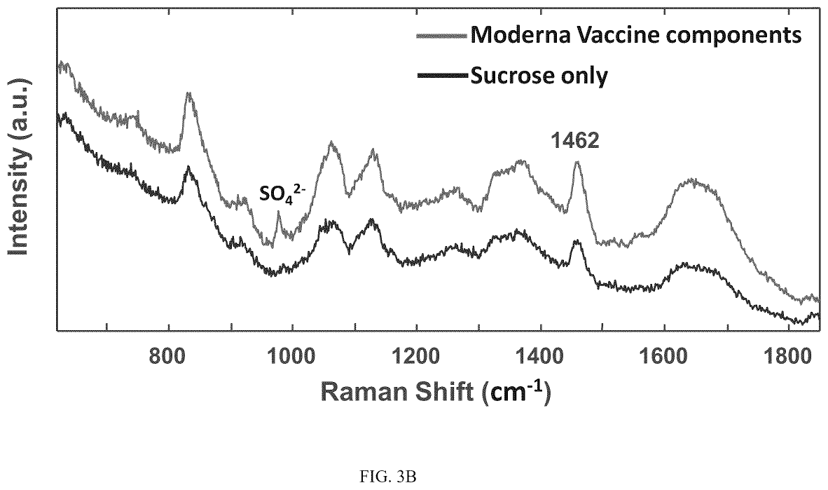

To further validate the applicability of the proposed method to actual COVID-19 vaccines, whether the lipids used in an RNA-based COVID vaccine sold by Moderna (hereinafter “Moderna vaccine lipids”) interfere with the Raman spectra of RNA at concentrations of lipids characteristic of the vaccine. Schoenmaker et al, mRNA-lipid nanoparticle COVID-19 vaccines: Structure and stability. International journal of pharmaceutics 2021, 601, 120586. As shown in FIG. 3A, the spectrum of the Moderna vaccine lipids at a concentration of 1.0 mM exhibits no Raman signal, indicating no resonance enhancement when excited at 266 nm. As shown in FIG. 3B, the buffer components in the Moderna vaccine,¹¹ (FDA. Moderna COVID-19 Vaccine Health Care Provider Fact Sheet. U.S. Food and Drug Administration, 2022.), specifically, Tris-HCl [pH 7.5], acetic acid, sodium acetate, and sucrose also showed no interference with the mRNA Raman signal. These results provide additional support for our hypothesis that DUVRR spectroscopy can directly probe the RNA in an mRNA vaccine.

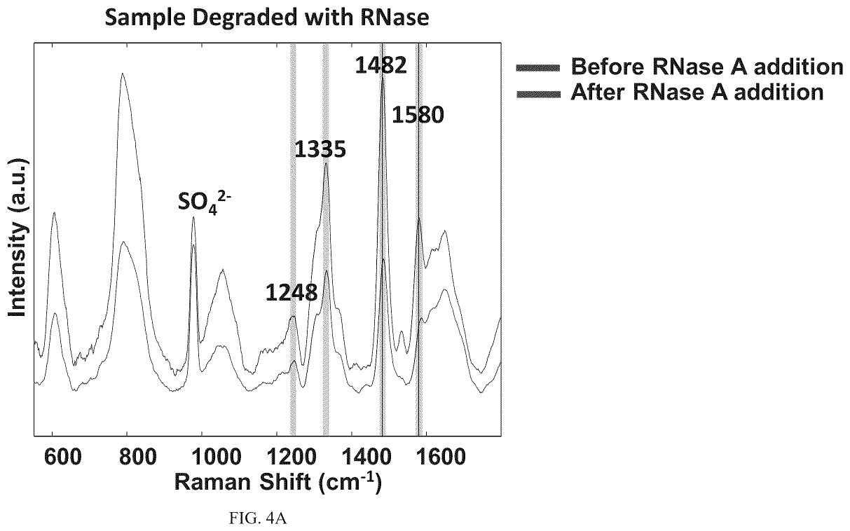

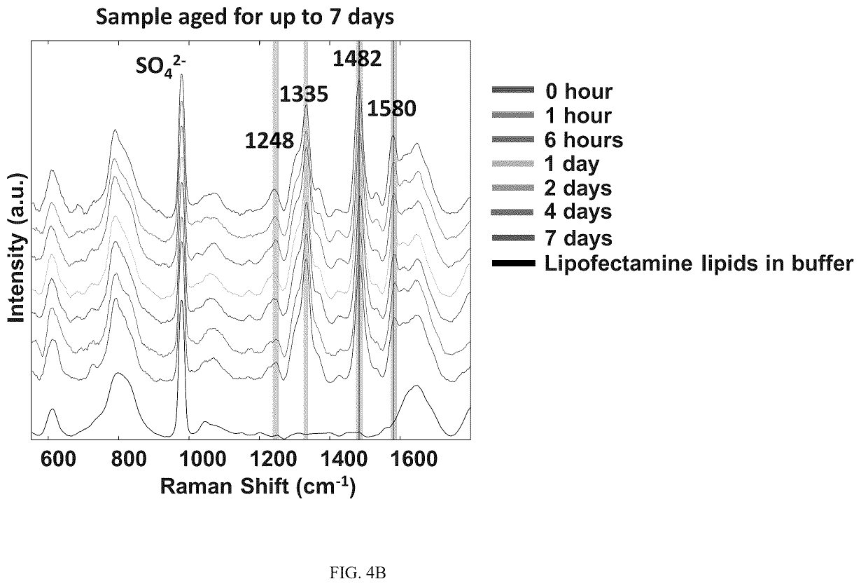

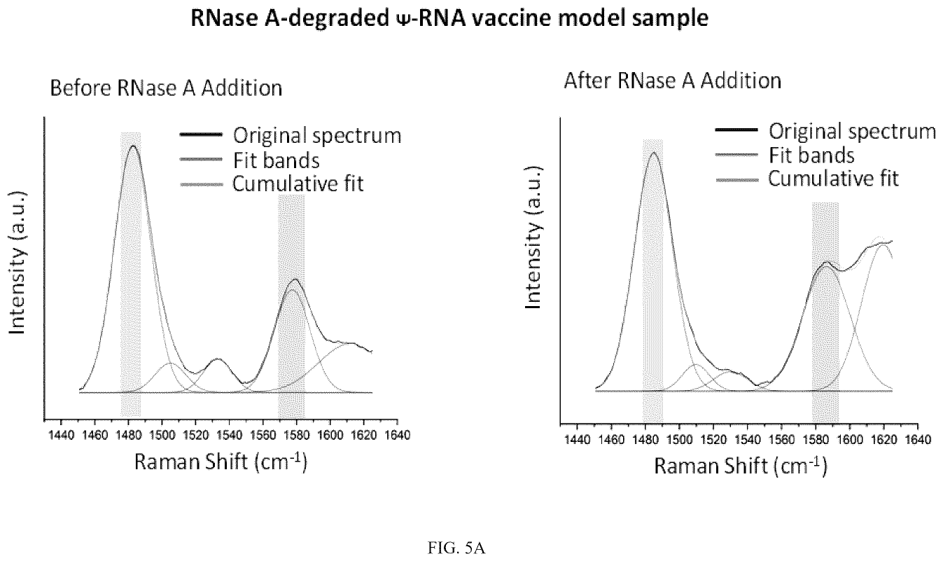

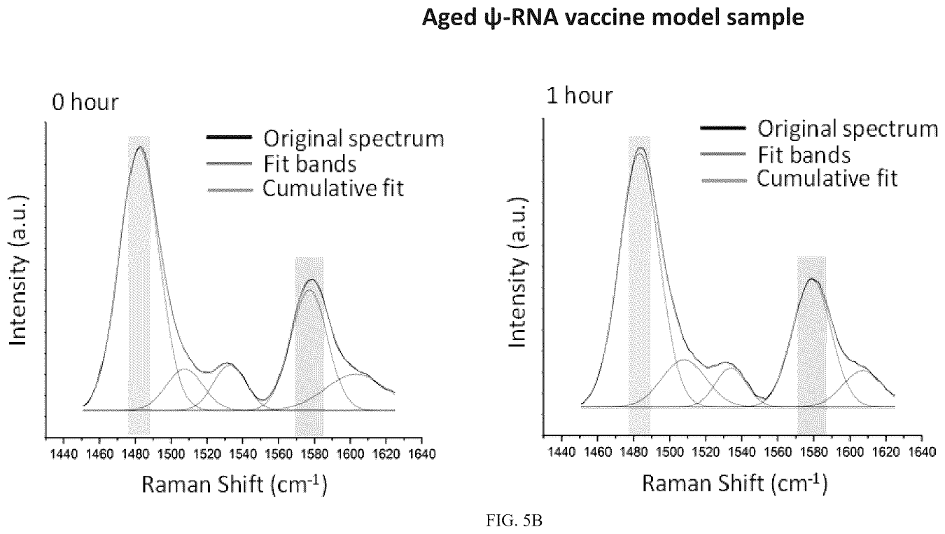

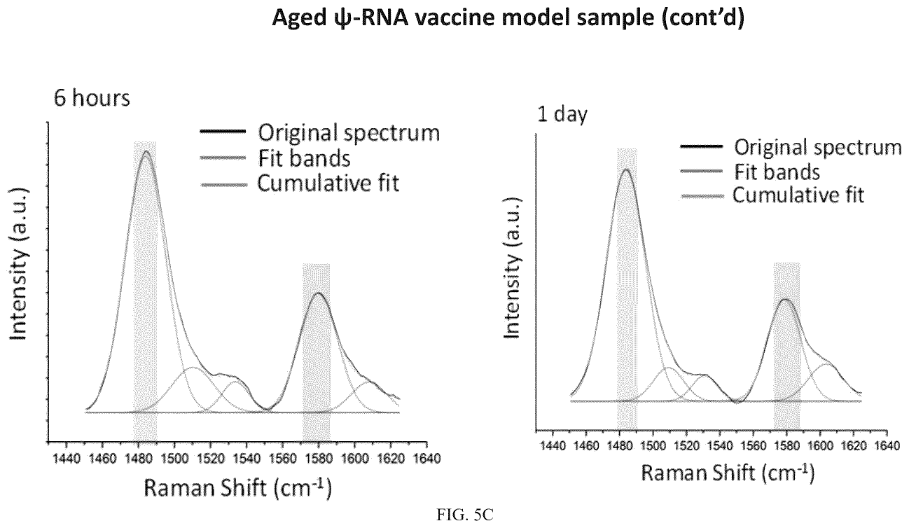

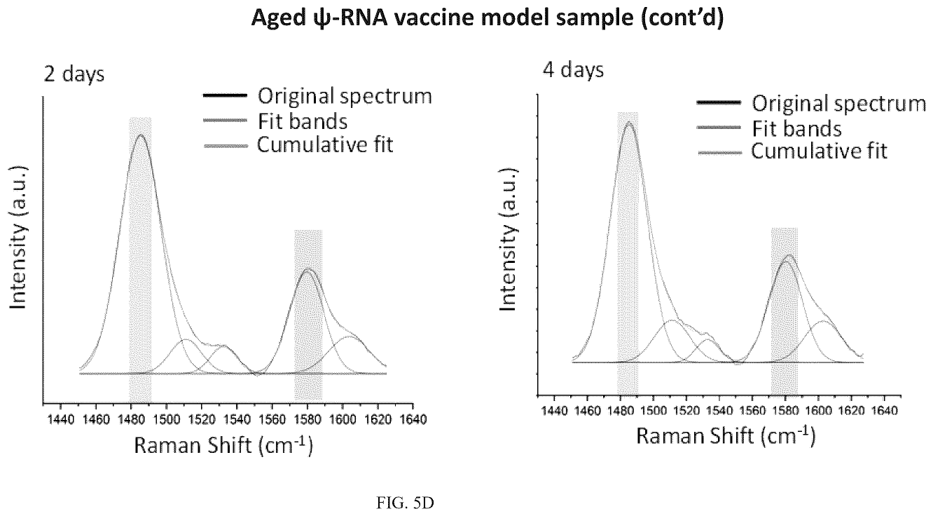

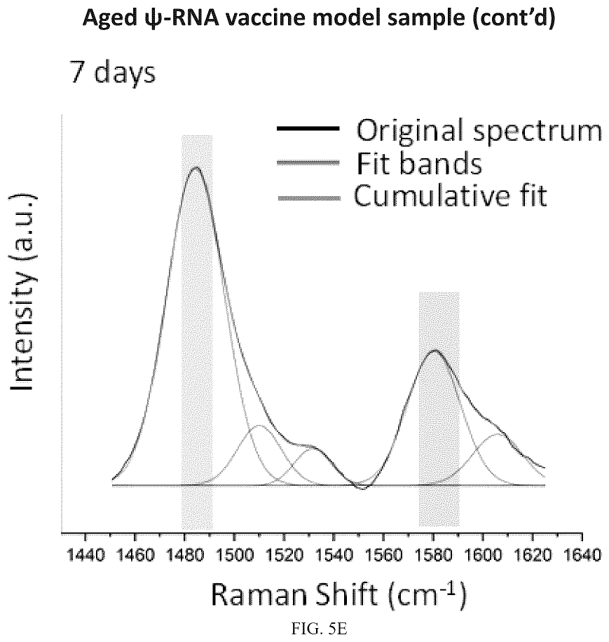

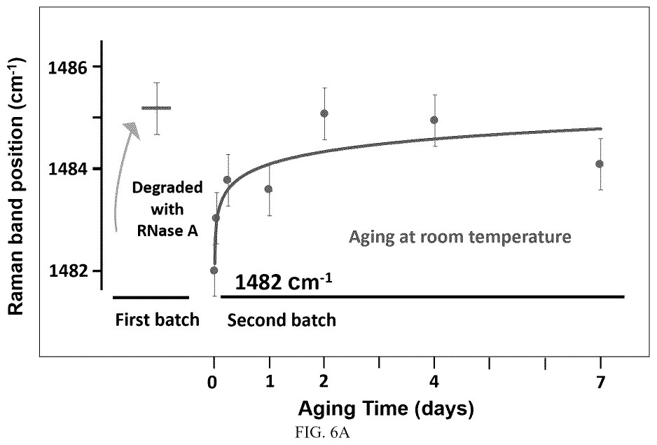

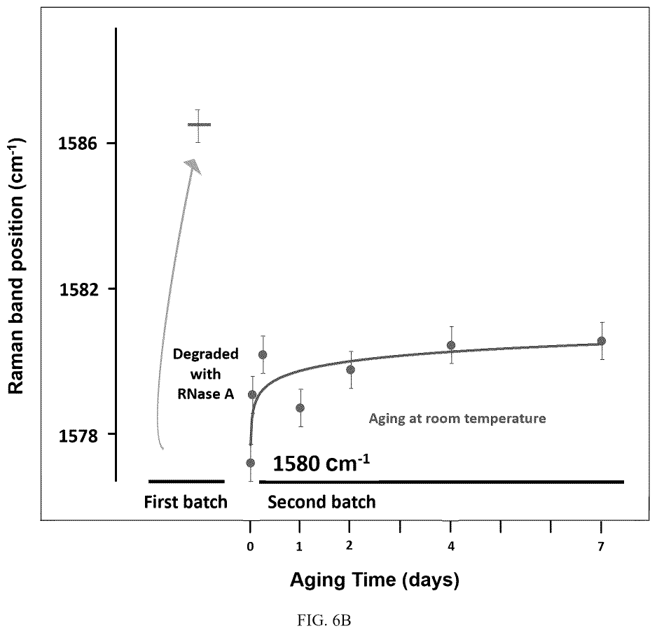

Subsequently, the degradation of RNA in the vaccine model was probed by DUVRR spectroscopy. The RNA in the vaccine model was degraded under two conditions, the addition of RNase A or aging of the vaccine model at room temperature for up to 7 days. FIGS. 4A-B show the DUVRR spectra collected for the vaccine model under these conditions. When large RNA molecules are degraded, such as luciferase RNA and the RNA used in COVID-19 vaccines, the secondary and tertiary structures of RNA may change. RNA base pairing and hydrogen bonds may be altered. Such perturbations are sensitive to DUVRR spectroscopy. Several Raman spectral markers associated with these changes are used here for the analysis of RNA degradation. FIG. 2B illustrates that variations in RNA Raman spectra under both degradation conditions follow a similar pattern. Bands at 1580 and 1482 cm⁻¹ are shifted to higher wavenumbers due to mRNA degradation. These bands do not overlap with spectra of the tested components of the Moderna vaccine as shown in FIG. 3B. After one day of incubation under ambient conditions, a substantial shift is noticed that is consistent with the gel electrophoresis results shown in FIG. 1C for aliquots of the same sample. Notably, the bands at 1580 and 1482 cm are the most intense in the DUVRR spectrum of the vaccine model, allowing for accurate determination of their band positions. Correspondingly, the band position can be monitored without fitting for practical applications and utilized to probe RNA degradation. However, the curve fitting procedure indicated that these band shifts are primarily attributable to 1580 and 1482 cm⁻¹ band shifts. FIGS. 5A-5E, for example, show multiple peak fitting of mRNA vaccine model spectra (1450-1625 cm⁻¹) using Gaussian peak function, and FIG. 6A-6B for example, show the shift of the (a) 1482- and (b) 1580-cm⁻¹ Raman bands based on multiple peak fitting procedure for mRNA vaccine model degraded with RNase A and aged for up to 7 days. In an embodiment, determining the Raman band shift may be more advantageous than assessing the Raman band intensity changes, such as in an example where because the latter requires precise calibration and an internal standard. In another example, monitoring the bands shift may offer more advantageous translation to different Deep UV instruments.

The shift of the band at 1580 cm⁻¹ to a higher wavenumber may indicate base unstacking due to the RNA structural perturbations. Fodor et al., Ultraviolet resonance Raman spectroscopy of the nucleotides with 266-, 240-, 218-, and 200-nm pulsed laser excitation. Journal of the American Chemical Society 1985, 107 (6), 1520-1529: Geng, J.; Aioub, M.; El-Sayed, M. A.: Barry. B. A. An Ultraviolet Resonance Raman Spectroscopic Study of Cisplatin and Transplatin Interactions with Genomic DNA. The Journal of Physical Chemistry B 2017, 121 (38), 8975-8983. The band at 1482 cm⁻¹ may shift to a higher wavenumber when guanine interacts with the 2,3-Dioleyloxy-N-[2(sperminecarboxamido)ethyl]-N,Ndimethyl-1-propanaminium trifluoroacetate (DOSPA) lipid. Matsui et al, Change of Poly(dG-dC)·Poly(dG-dC) in Cationic Polyamine Liposome Complexes: Effect of Charge Density and Flexibility of Amine Chains in Headgroups. The Journal of Physical Chemistry B 2000, 104 (37), 8871-8875. DOSPA is one of the lipids utilized in lipofectamine. DUVRR spectroscopy is therefore sensitive to changes in the RNA structure and its interaction with lipids in vaccines.

As disclosed herein, a method of detecting degradation of a polynucleotide may include performing DUVRR spectroscopy thereupon, including RNA vaccines in situ. Due to resonance enhancement, the contribution from RNA at 266-nm excitation dominates the Raman spectra of the mRNA vaccine. The observed spectral changes due to the RNA degradation are consistent with the gel electrophoresis results. A DUVRR spectroscopic method of detecting degradation of a polynucleotide as disclosed herein may be applied automatically for a nondestructive evaluation of the integrity of polynucleotides, including but not limited to RNA vaccines and for probing RNA integrity during the development and production of RNA-based materials using a portable instrument, such as the one currently used on Mars. Beegle et al., SHERLOC: Scanning habitable environments with Raman & luminescence for organics & chemicals. In 2015 IEEE Aerospace Conference, 7-14 Mar. 2015, 2015; pp 1-11. In situ analysis of polynucleotide degradation using deep-UV excitation may be performed where the polynucleotide is contained in, for example, pharmaceutical packaging materials of fused quartz and plastic (such as those that are commercially available, from sources such as Honeywell and Momentive Technologies).^{20,21}

Although some non-limiting examples have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

### REFERENCES

(1) Hogan, M. J.; Pardi, N. mRNA Vaccines in the COVID-19 Pandemic and Beyond. Annual Review of Medicine 2022, 73 (1), 17-39. DOI: 10.1146/annurev-med-042420-112725.
(2) Uddin, M. N.: Roni, M. A. Challenges of Storage and Stability of mRNA-Based COVID-19 Vaccines. Vaccines 2021, 9 (9), 1033, DOI: 10.3390/vaccines9091033.
(3) Schoenmaker, L.; Witzigmann, D.; Kulkarni, J. A.; Verbeke, R.; Kersten, G.; Jiskoot, W.; Crommelin, D. J. A. mRNA-lipid nanoparticle COVID-19 vaccines: Structure and stability. International journal of pharmaceutics 2021, 601, 120586. DOI: 10.1016/j.ijpharm.2021.120586.
(4) WHO. Evaluation of the quality, safety and efficacy of messenger RNA vaccines for the prevention of infectious diseases: Regulatory considerations. 2021.
(5) Fodor, S. P. A.; Rava, R. P.; Hays, T. R.; Spiro, T. G. Ultraviolet resonance Raman spectroscopy of the nucleotides with 266-, 240-, 218-, and 200-nm pulsed laser excitation. Journal of the American Chemical Society 1985, 107 (6), 1520-1529. DOI: 10.1021/ja00292a012.
(6) John, N.; George, S. Chapter 5—Raman Spectroscopy. In Spectroscopic Methods for Nanomaterials Characterization, Thomas, S., Thomas. R., Zachariah, A. K., Mishra, R. K. Eds.; Elsevier, 2017; pp 95-127.
(7) Fodor, S. P. A.; Spiro, T. G. Ultraviolet resonance Raman spectroscopy of DNA with 200-266-nm laser excitation. Journal of the American Chemical Society 1986, 108 (12), 3198-3205. DOI: 10.1021/ja00272a006.
(8) Rossi, B.; Tortora, M.; Catalini, S.: Vigna, J.; Mancini, I.: Gessini, A.; Masciovecchio, C.; Mele, A. Insight into the thermal stability of DNA in hydrated ionic liquids from multi-wavelength UV resonance Raman experiments. Physical Chemistry Chemical Physics 2021, 23 (30), 15980-15988, 10.1039/D1CP01970H. DOI: 10.1039/D1CP01970H.
(9) Geng, J.; Aioub, M.; El-Saved, M. A.; Barry, B. A. An Ultraviolet Resonance Raman Spectroscopic Study of Cisplatin and Transplatin Interactions with Genomic DNA. The Journal of Physical Chemistry B 2017, 121 (38), 8975-8983. DOI: 10.1021/acs.jpcb.7b08156.
(10) Andries, O.; Mc Cafferty, S.: De Smedt, S. C.: Weiss, R.; Sanders, N. N.; Kitada, T. N(1)-methylpseudouridine-incorporated mRNA outperforms pseudouridine-incorporated mRNA by providing enhanced protein expression and reduced immunogenicity in mammalian cell lines and mice. Journal of controlled release: official journal of the Controlled Release Society 2015, 217, 337-344. DOI: 10.1016/j.jconrel.2015.08.051.
(11) FDA. Moderna COVID-19 Vaccine Health Care Provider Fact Sheet. U.S. Food and Drug Administration, 2022.
(12) Sciolino, N.; Reverdatto, S.; Premo, A.; Breindel, L.; Yu, J.; Theophall, G.; Burz, D. S.; Liu, A.: Sulchek, T.: Schmidt, A. M.; et al. Messenger RNA in lipid nanoparticles rescues HEK 293 cells from lipid-induced mitochondrial dysfunction as studied by real time pulse chase NMR. RTPC-NMR, spectroscopy. Scientific Reports 2022, 12 (1), 22293. DOI: 10.1038/s41598-022-26444-z.
(13) Aranda, P. S.; LaJoie, D. M.; Jorcyk, C. L. Bleach gel: a simple agarose gel for analyzing RNA quality. Electrophoresis 2012, 33 (2), 366-369. DOI: 10.1002/elps.201100335 From NLM.
(14) Lednev, I. K.; Ermolenkov, V. V.; He, W.; Xu, M. Deep-UV Raman spectrometer tunable between 193 and 205 nm for structural characterization of proteins. Analytical and Bioanalytical Chemistry 2005, 381 (2), 431437. DOI: 10.1007/s00216-004-2991-5.
(15) Karikó, K.; Muramatsu, H.; Welsh, F. A.; Ludwig, J.; Kato, H.; Akira, S.; Weissman, D. Incorporation of pseudouridine into mRNA yields superior nonimmunogenic vector with increased translational capacity and biological stability. Mol Ther 2008, 16 (11), 1833-1840. DOI: 10.1038/mt.2008.200 From NLM.
(16) Démoulins, T.: Englezou, P. C.: Milona, P.; Ruggli, N.; Tirelli, N.: Pichon, C.; Sapet, C.: Ebensen, T.; Guzman, C. A.; McCullough, K. C. Self-Replicating RNA Vaccine Delivery to Dendritic Cells. Methods Molecular Biology 2017, 1499, 37-75. DOI: 10.1007/978-1-4939-6481-9_3.
(17) Matsui. H.; Pan, S. Conformation Change of Poly(dG-dC)·Poly(dG-dC) in Cationic Polyamine Liposome Complexes: Effect of Charge Density and Flexibility of Amine Chains in Headgroups. The Journal of Physical Chemistry B 2000, 104 (37), 8871-8875. DOI: 10.1021/jp001439g.
(18) Godbey, W. In An introduction to biotechnology: the science, technology and medical applications, 1st ed.; Academic Press, 2014; pp 275-312
(19) Beegle, L.: Bhartia, R.; White, M.: DeFlores, L.; Abbey, W.; Yen-Hung, W.; Cameron, B.; Moore, J.; Fries, M.; Burton, A.; et al. SHERLOC: Scanning habitable environments with Raman & luminescence for organics & chemicals. In 2015 IEEE Aerospace Conference, 7-14 Mar. 2015, 2015; pp 1-11. DOI: 10.1109/AERO.2015.7119105.
(20) Kansteiner, F. Honeywell adds COVID vaccine-ready vials to its new line of glass alternatives. 2020. https://www.fiercepharma.com/drug-delivery/honeywell-answers-call-for-drug-vials-glass-alternative-aclar-edge (accessed 2023 August, 24).
(21) MomentiveTechnologies. QUARTZ VIALS. https://www.momentivetech.com/products/quartz/quartz-vials/ (accessed 2023 August, 24).

What is claimed is:

1. A method of detecting degradation of a polynucleotide, comprising
irradiating the polynucleotide with ultraviolet light having a wavelength within a range of from about 200 nm to about 280 nm, and
detecting Raman scattering by the polynucleotide.

2. The method of claim 1, wherein the polynucleotide comprises RNA.

3. The method of claim 1 or 2, wherein the polynucleotide comprises mRNA.

4. The method of any one of claims 1 through 3, wherein the polynucleotide comprises one or more of N1-methylpseudouridine substituted for uridine, pseudouridine substituted for uridine, and 5-methylcytidine substituted for cytidine.

5. The method of any one of claims 1 through 4, wherein the polynucleotide comprises N1-methylpseudouridine substituted for uridine.

6. The method of any one of claims 1 through 5, wherein the range is from about 230 nm to about 280 nm.

7. The method of any one of claims 1 through 6, wherein the range is from about 260 nm to about 270 nm.

8. The method of any one of claims 1 through 7, wherein the wavelength is about 266.

9. The method of any one of claims 1 through 8, wherein the polynucleotide comprises a vaccine, an antisense oligonucleotide, or a micro RNA.

10. The method of any one of claims 1 through 9, wherein the polynucleotide is in a lipid nanoparticle.

11. The method of any one of claims 1 through 10, wherein the Raman scattering comprises a wavenumber shift of one or more Raman band of at least 1 cm⁻¹.

12. The method of any one of claims 1 through 11, wherein the Raman scattering comprises a wavenumber shift of one or more Raman band of at least 2 cm⁻¹.

13. The method of any one of claims 1 through 12, wherein the Raman scattering comprises a wavenumber shift of one or more Raman band of at least 3 cm⁻¹.

14. The method of any one of claims 1 through 13, wherein the Raman scattering comprises a wavenumber shift of a Raman band at 1580 cm⁻¹.

15. The method of any one of claims 1 through 14, wherein the Raman scattering comprises a wavenumber shift of a Raman band at 1482 cm⁻¹.

16. The method of any one of claims 1 through 15, wherein the Raman scattering comprises a decrease in intensity of one or more Raman band.

17. The method of any one of claims 1 through 16, wherein the Raman scattering comprises a decrease in intensity of a Raman band at 1580 cm⁻¹.

18. The method of any one of claims 1 through 17, wherein the Raman scattering comprises a decrease in intensity of a Raman band at 1482 cm⁻¹.

19. A method of detecting degradation of a test polynucleotide, comprising
irradiating a control polynucleotide and the test polynucleotide with ultraviolet light having a wavelength within a range of from about 200 nm to about 280 nm,
detecting Raman scattering by the control polynucleotide and by the test polynucleotide, and
detecting a difference between the Raman scattering by the test polynucleotide and by the control polynucleotide.

20. The method of claim 19, wherein the test polynucleotide and the control polynucleotide comprise RNA.

21. The method of claim 19 or 20, wherein the test polynucleotide and the control polynucleotide comprise mRNA.

22. The method of any one of claims 19 through 21, wherein the test polynucleotide and the control polynucleotide comprise one or more of N1-methylpseudouridine substituted for uridine, pseudouridine substituted for uridine, and 5-methylcytidine substituted for cytidine.

23. The method of any one of claims 19 through 22, wherein the test polynucleotide and the control polynucleotide comprise N1-methylpseudouridine substituted for uridine.

24. The method of any one of claims 19 through 23, wherein the range is from about 230 nm to about 280 nm.

25. The method of any one of claims 19 through 24, wherein the range is from about 260 nm to about 270 nm.

26. The method of any one of claims 19 through 25, wherein the wavelength is about 266.

27. The method of any one of claims 19 through 26, wherein the test polynucleotide and the control polynucleotide comprise a vaccine, an antisense oligonucleotide, or a micro RNA.

28. The method of any one of claims 19 through 27, wherein the test polynucleotide and the control polynucleotide are in lipid nanoparticles.

29. The method of any one of claims 19 through 28, wherein the difference between the Raman scattering by the test polynucleotide and by the control polynucleotide comprises a wavenumber shift of one or more Raman band of at least 1 cm⁻¹.

30. The method of any one of claims 19 through 29, wherein the difference between the Raman scattering by the test polynucleotide and by the control polynucleotide comprises a wavenumber shift of one or more Raman band of at least 2 cm⁻¹.

31. The method of any one of claims 19 through 30, wherein the difference between the Raman scattering by the test polynucleotide and by the control polynucleotide comprises a wavenumber shift of one or more Raman band of at least 3 cm⁻¹.

32. The method of any one of claims 19 through 31, wherein the difference between the Raman scattering by the test polynucleotide and by the control polynucleotide comprises a wavenumber shift of a Raman band at 1580 cm⁻¹.

33. The method of any one of claims 19 through 32, wherein the difference between the Raman scattering by the test polynucleotide and by the control polynucleotide comprises a wavenumber shift of a Raman band at 1482 cm⁻¹.

34. The method of any one of claims 19 through 33, wherein the difference between the Raman scattering by the test polynucleotide and by the control polynucleotide comprises a decrease in intensity of one or more Raman band.

35. The method of any one of claims 19 through 34, wherein the difference between the Raman scattering by the test polynucleotide and by the control polynucleotide comprises a decrease in intensity of a Raman band at 1580 cm⁻¹.

36. The method of any one of claims 19 through 35, wherein the difference between the Raman scattering by the test polynucleotide and by the control polynucleotide comprises a decrease in intensity of a Raman band at 1482 cm⁻¹.

37. A method of detecting degradation of an RNA polynucleotide, comprising
irradiating the polynucleotide with ultraviolet light having a wavelength within a range of from about 260 nm to about 270 nm, and
detecting Raman scattering by the polynucleotide.

38. The method of 37, wherein the Raman scattering comprises a wavenumber shift of one or more Raman band of at least 2 cm⁻¹.

39. The method of claim 37 or 38, wherein the Raman scattering comprises a wavenumber shift of a Raman band at 1580 cm⁻¹.

40. The method of claim 37 or 38, wherein the Raman scattering comprises a wavenumber shift of a Raman band at 1482 cm⁻¹.

41. A method of detecting degradation of a test polynucleotide, comprising
irradiating a control polynucleotide and the test polynucleotide with ultraviolet light having a wavelength within a range of from about 260 nm to about 270 nm,
detecting Raman scattering by the control polynucleotide and by the test polynucleotide, and
detecting a difference between the Raman scattering by the test polynucleotide and by the control polynucleotide.

42. The method of claim 41, wherein the Raman scattering comprises a wavenumber shift of one or more Raman band of at least 2 cm⁻¹.

43. The method of claim 41 or 42, wherein the Raman scattering comprises a wavenumber shift of a Raman band at 1580 cm⁻¹.

44. The method of claim 41 or 42, wherein the Raman scattering comprises a wavenumber shift of a Raman band at 1482 cm⁻¹.

45. The method of any one of claims 1, 4, and 6-18, wherein the polynucleotide comprises DNA.

46. The method of any one of claims 19, 22, and 24-36, wherein the test polynucleotide and the control polynucleotide comprise DNA.

47. The method of any one of claims 37-40, wherein the polynucleotide comprises DNA.

48. The method of any one of claims 41-44, wherein the test polynucleotide and the control polynucleotide comprise DNA.
